(19) United States
(12) Patent Application Publication
(10) Pub. No.: US 2022/0249607 A1
(43) Pub. Date: Aug. 11, 2022

(54) Method of treatment of degenerative diseases caused by membrane channel-forming peptides fragments

(71) Applicants: Dmitry Zaretsky, Chapel Hill, NC (US); Maria Zaretskaia, Chapel Hill, NC (US)

(72) Inventors: Dmitry Zaretsky, Chapel Hill, NC (US); Maria Zaretskaia, Chapel Hill, NC (US)

(73) Assignee: Zarbio, Chapel Hill, NC (US)

(21) Appl. No.: 17/666,021

(22) Filed: Feb. 7, 2022

Related U.S. Application Data
(60) Provisional application No. 63/199,932, filed on Feb. 3, 2021.

(51) Int. Cl. A61K 38/17

(52) U.S. Cl. CPC A61K 38/1716

(57) ABSTRACT

The present invention provides the method to prevent or slow down the progression of degenerative diseases caused by membrane channel-forming peptides. For many of these diseases, there is no known treatment based on the etiology and pathogenesis of the corresponding disease. Until recently, there was no integrative theory explaining multiple symptoms and observations associated with such diseases. In response to this challenge, we developed the amyloid degradation toxicity theory of Alzheimer's disease (AD). Within this concept, the etiology of the disease is the formation of beta-amyloid fragments which form membrane channels. We claim that the stopping the production of toxic fragments by inhibiting biochemical pathways producing channel-forming fragments (for example, by protease inhibitors) will prevent or slow down the progression AD. Also, we claim that the same molecular mechanism is involved in multiple neurodegenerative diseases and diabetes type II, so the invented method can be used to treat them.

### CROSS-REFERENCES TO RELATED U.S. APPLICATION DATA

Provisional application No. 63/199,982, filed on Feb. 6, 2021

### STATEMENT REGARDING FEDERALLY SPONSORED RESEARCH OR DEVELOPMENT

This invention was made without government support.

### TECHNICAL FIELD OF INVENTION

Present invention relates to the methods of treatment of diseases caused by proteins, which can undergo a misfolding after synthesis. Immediately after the synthesis, these proteins are soluble, but after misfolding, protein molecules aggregate and form clumps of insoluble protein (plaques) in various tissues. The clumps are easily identified by histological staining for amyloid; therefore, these diseases are often referred to as amyloid diseases. The major content of insoluble material in the clump is polymeric peptides bound by intra- and intermolecular hydrogen bonds. Despite being a hallmark of neurodegenerative diseases, these clumps by themselves are not toxic and are not considered the major reason of neuronal and cellular toxicity and death. In the process of polymerization, peptides form oligomers which are considered the culprits of toxicity.

The toxicity of oligomeric amyloidogenic peptides is mediated, at least in part, by the formation of barrel-like structures, which have a central hole and incorporate into cellular membranes. Membrane channels (also called pores) pass various ions and can be large enough to leak macromolecules such as proteolytic enzymes. Short fragments of beta-amyloid are incomparably more potent in permeabilizing lipid membranes than full-length peptide. To be channel-forming, peptide fragments need to carry positive charge, consist of mostly hydrophobic residues, and form beta-sheet.

Based on amyloid channel concept, we have developed the amyloid degradation toxicity theory of Alzheimer's disease. Intralysosomal proteolysis of beta-amyloid appears to be the etiological process, which results in the formation of peptides permeabilizing lysosomal membrane. Leaking lysosomal proteases lead to cell death through necrosis or apoptosis. Other proteins which are believed to be involved into other degenerative disease (such as superoxide dismutase and amylin) also contain peptide sequences, which have features required for channel formation. This allows to extend the amyloid degradation toxicity theory to the etiology and pathogenesis of other degenerative diseases.

We claim the method to prevent or treat degenerative diseases which are caused by misfolding proteins. The method consists of inhibition of enzymes which degrade longer peptides into fragments forming membrane pores. The list of diseases which can be treated by this novel class of pharmacological agents includes, but is not limited to, diseases caused by beta-amyloid or tau protein (e.g. Alzheimer's disease), by alpha-synuclein (e.g. Parkinson's disease), by huntingtin (e.g. Huntington's disease), by TDP-43 protein or superoxide dismutase (e.g. Amyotrophic Lateral Sclerosis), by amylin (e.g. diabetes Type II). The list of diseases can also include prion diseases, Creutzfeldt-Jakob disease, alcoholism, brain and spinal cord injury, retinal degeneration, including age-associated retinal degeneration, neurodegeneration caused by psychostimulants (including amphetamine and its derivatives), endotoxic shock, and chronic traumatic encephalopathy.

### BACKGROUND OF THE INVENTION

### Misfolding Peptides, Amyloidogenesis, and Cellular/Neuronal Degeneration

Diseases caused by misfolded proteins vary significantly. However, they have a common feature: immediately after the synthesis the protein has no secondary or tertiary structure and is soluble, but under various conditions it may undergo conformational changes, which ultimately result in the formation of beta-sheets and, in the loss of the solubility after polymerization. Individual molecules with intramolecular beta-sheet structure are linked to other such molecules forming protofibrils. Protofibrils tend to aggregate and attract other molecules with relatively low solubility. As a result, insoluble conglomerates become large enough to be visible after histochemical staining of tissue sections. This was how these diseases were identified and grouped as amyloid diseases—various methods of staining reveal amorphous clumps of substance in brain or other tissues. Importantly, there was a correlation between where the clumps could be observed with clinical observations—dopaminergic areas contained such clumps in Parkinson's disease, while cortical areas are prone to the accumulation of clumps in Alzheimer's disease. Appearance of inclusions usually was accompanied by the disappearance of cells, such as dopaminergic neurons (Parkinson's disease) or cortical cells (Alzheimer's disease). Such correlation prompted early theory that the insoluble substance is the cause of the disease.

With time, accumulated observations showed that clinical severity of disease is not dependent on the number or the size of such inclusions. Importantly, the expression of inclusions has much better correlation with the length of disease than with the severity. Even more, the presence of inclusions does not necessarily result in the presence of the disease—highly expressed inclusions can be observed in medically healthy patients. However, there was strong correlation between the disappearance of neurons and clinical outcome. This led to the understanding that insoluble protein could be just another consequence of some process which is also responsible for cellular death.

Major promise to finding the cure for this group of diseases is in the comprehension of the process, which underlies the formation of insoluble protein inclusions, and the relationship of this process to the cellular death. Preventing cellular death is the only way to treat, delay the onset or slow down the progression of these diseases. Together with preventative screening and/or early diagnosis, such treatment can be a way to eradicate neurodegenerative diseases.

As it was mentioned above, freshly synthesized polypeptides do not have fixed conformation and are water-soluble. Over time, some molecules develop hydrogen bonds which fix specific turns and form beta-sheets, one of major secondary protein structures. Intramolecular hydrogen bonds fix turns within the molecules (label 1 at the FIG. 4), while intermolecular bonds attach multiple polypeptide molecules to each other (label 2 at the FIG. 4) forming oligomers (label 3 at the FIG. 4). Structure-wise, protofibrils are formed by core pleated beta-sheet structures with short peptide tails spreading to the sides of the core. Interaction between protofibrils results in the formation of large fibrils, the process which may also include other proteins (label 5 at the FIG. 4) which become stuck on the protofibrils and remain trapped in the insoluble protein clumps.

It became wide-accepted that cellular or neuronal toxicity is mediated by oligomeric structures, while soluble monomers and formed insoluble large-size fibrils appear mostly non-toxic. Considering that oligomers and fibrils are in equilibrium, the addition of “purified” formed fibrils in experimental settings also results in the formation of some level of oligomers, so even in strict experimental settings it is difficult to identify if cellular/neuronal toxicity is caused by administered fibrils or smaller oligomers.

Multiple biochemical pathways were hypothesized as the causes of neuronal death induced by amyloid. Numerous reviews mention the activation of apoptosis, an increase in oxidative stress, ion disturbances, immune system involvement, and more. However, most mechanisms don't name a molecular interaction that initiates biochemical and biophysical pathways leading to cell death. In pharmacological terms—no primary molecular action is identified. It is typical for the toxicology of exogenous substances to expect that Aβ requires some other protein (such as a receptor or ion channel) to be involved in exerting toxic action. Various molecular targets of amyloid were extensively reviewed (Smith and Strittmatter 2017, Mroczko, Groblewska et al. 2018). However, Aβ provides a mechanism that does not require the presence of any other protein (Glabe 2006).

In the early 1990s, using electrophysiological techniques several groups independently demonstrated that it is possible to detect the formation of ion channels in lipid membranes exposed to either Aβ₁₋₄₀, or Aβ₁₋₄₂, or its short fragment (Aβ₂₅₋₃₅) (Arispe, Pollard et al. 1993, Arispe, Rojas et al. 1993, Simmons and Schneider 1993, Arispe, Pollard et al. 1994, Arispe, Pollard et al. 1994, Durell, Guy et al. 1994, Mirzabekov, Lin et al. 1994, Pollard, Arispe et al. 1995, Arispe, Pollard et al. 1996, Rhee, Quist et al. 1998, Hirakura, Lin et al. 1999, Lin, Zhu et al. 1999, Lin, Bhatia et al. 2001, Lin and Kagan 2002, Alarcon, Brito et al. 2006, Arispe, Diaz et al. 2007, Jang, Arce et al. 2010, Bode, Baker et al. 2017). Ion channels were reproducibly formed if the peptide was mixed with lipids during the formation of membranes. Furthermore, if the peptide was added to already-formed lipid membranes, the ion channels appeared very quickly—within minutes at most. Since these first discoveries, the amyloid channel theory has become one of the major theories explaining the development of Alzheimer's disease (Pollard, Rojas et al. 1993, Arispe, Pollard et al. 1994, Pollard, Arispe et al. 1995, Arispe, Diaz et al. 2007, Shirwany, Payette et al. 2007, Diaz, Simakova et al. 2009, Jang, Arce et al. 2010, Jang, Connelly et al. 2013).

### Properties of Amyloid Channels

Amyloid channels formed by beta-amyloid pass all the studied cations—sodium, potassium, calcium, cesium, and lithium (Arispe, Rojas et al. 1993). The permeability of channels to ions that are most critical for neurons are not equal, specifically P_{Ca}²⁺=P_{K}⁻>P_{Na}⁺, but the permeability to calcium is only 30% higher than to sodium (Arispe, Rojas et al. 1993). In another study, the ratio was higher P_{Ca}²⁺:P_{K}⁺P_{Na}⁺:P_{Cl}⁻=5.4:1.6:1.4:1 (Mirzabekov, Lin et al. 1994). However, in any case, the difference does not justify the exclusivity of interest to calcium. In fact, disturbances of the intracellular concentrations of major cations induced by the application of Aβ mirror each other—once the cellular membrane opens for one ion, others start flowing across (Abramov, Canevari et al. 2004). Lysosomes of cells exposed to Aβ leak membrane-impermeant anionic dye Lucifer Yellow (MW 444) (Yang, Chandswangbhuvana et al. 1998). Importantly, fluxes of calcium and pH were synchronous when recorded simultaneously in cells exposed to Aβ (Abramov, Canevari et al. 2004), so we can assume that non-specificity of amyloid-induced membrane permeabilization applies to protons as well.

Membrane permeabilization occurs when negatively charged membranes are exposed to the positively charged amyloid fragments, such as Aβ₂₅₋₃₅ (Zaretsky and Zaretskaia 2020), which is known to create beta-sheets and aggregate (Naldi, Fiori et al. 2012). A similar peptide without a positive charge, Aβ₂₂₋₃₅, does not create a noticeable number of channels. Also, Aβ₂₅₋₃₅ does not permeabilize liposomes made of neutral phosphatidylcholine (Zaretsky and Zaretskaia 2020). The difference of effects between phospholipids and fragments with a different charge confirms the role of electrostatic interactions, which was also demonstrated in other studies (Alarcon, Brito et al. 2006).

The full-length peptide Aβ₁₋₄₂ is ineffective in the permeabilization of the membranes in our experiments (Zaretsky and Zaretskaia 2020). Our observations match the data of Mirzabekov et al, who showed that in reasonably low concentrations, only the fragment Aβ₂₅₋₃₅, but not full-length Aβ₁₋₄₀ or Aβ₁₋₄₂, is able to create channels (Mirzabekov, Lin et al. 1994).

Amyloid-formed channels (ACs) are quite different from typical specialized ion channels, such as sodium or potassium channels. ACs have multiple conductance states and demonstrate high- and low-frequency transitioning between these states. Also, the range of measured conductance is very wide even within the same study. However, the most unique feature of these channels is the absolute values of their conductance—a single channel can have a conductance of up to several nanosiemens, while the conductance of a typical ion channel (such as a sodium channel) is measured in picosiemens (Arispe, Pollard et al. 1993). All features point to supramolecular barrel-shaped structures formed by multiple peptide molecules, as was first modeled by Durell et al (Durell, Guy et al. 1994). The need for aggregation appears to be in line with the role of oligomers in Aβ toxicity (Teplow 2013, Cline, Bicca et al. 2018).

### Exposure to Beta-Amyloid Induces Lysosomal Dysfunction and Permeabilization

When cells are exposed to the beta-amyloid, it is accumulated intracellularly (Knauer, Soreghan et al. 1992). The process of the internalization of Aβ occurs through endocytosis (Jin, Kedia et al. 2016, Wesen, Jeffries et al. 2017, Heckmann, Teubner et al. 2019). In general, endosomes merge with lysosomes to digest the taken extracellular content (He and Klionsky 2009, Yin, Pascual et al. 2016). This mechanism readily explains how the amyloid peptide accumulates in lysosomes (Marshall, Vadukul et al. 2020). However, the reason for which AD is associated with an accumulation of undigested peptide in dysfunctional lysosomes, remains unexplained.

The dramatically increased presence of autophagic vacuoles is one of the features of Alzheimer's disease (Nixon, Wegiel et al. 2005). It is notable that dystrophic swellings induced by lysosomal proteolysis inhibition appear in dendrites. Historically, it was considered that lysosomes are formed in the neuronal soma, while autophagosomes are created where needed, including axons and dendrites of neurons, and are carried to the soma for processing (Cherra and Chu 2008). However, autophagosomes can fuse with lysosomes while transported along microtubules to the cell body, or autophagy can be carried out completely at the cell's periphery (Ariosa and Klionsky 2016). The dynamic of immunochemical markers also supports the notion that neurite dystrophy evolves from dysfunctions of pre-autophagosomes (Sharoar, Hu et al. 2019).

One of symptoms of lysosomal disfunction is permeabilization of membranes. The permeabilization of lysosomes by amyloid and even the leakage of lysosomal content can be visualized. Ji et al., 2002 allowed cells to accumulate membrane-impermeant Lucifer Yellow, which enters the cell through endocytosis (Ji, Miranda et al. 2002). In untreated cells, fluorescent objects were observed as small, circumscribed vesicular structures resembling intact lysosomes creating a punctate pattern of fluorescence. After treatment with Aβ₁₋₄₂, however, it was readily apparent that cells displayed a diffuse intracellular pattern of fluorescence. In these experiments, the incubation time with amyloid was 20 hours (Ji, Miranda et al. 2002), a period sufficient enough for an endocytic uptake and processing of the exogenously added peptide. Such observations confirm that an exposure to amyloid peptide makes lysosomes permeable to relatively large compounds such as Lucifer Yellow (MW 444).

Importantly, the lysosomal disfunction can be the result of enzymatic failure. It was long noted that dystrophic swellings induced by lysosomal proteolysis inhibition resemble those in AD brains and in mouse models of AD (Boland, Kumar et al. 2008). In experiments, lysosomal proteolysis can be disrupted by either a direct cathepsin inhibition or a suppression of lysosomal acidification. The inhibition of proteolysis in lysosomes slows the axonal transport of autolysosomes, late endosomes, and lysosomes, and causes their selective accumulation within dystrophic axonal swellings, despite the axonal transport system being preserved (Lee, Sato et al. 2011). Importantly, when experimental inhibition of lysosomal proteolysis is reversed, autophagic substrates are cleared and the axonal dystrophy disappears (Lee, Sato et al. 2011).

Finally, the lysosomal membrane carries a significant negative charge due to a presence of bis(monoacylglycero)phosphate which represents up to a quarter of total lipid making lysosomal membrane. This key characteristic makes lysosomal membrane a reasonable target for permeabilization by amyloid channels. Much more importantly, these organelles are unique in the cellular machinery due to their role in protein degradation. Membrane channels are created by amyloid fragments, but not a full-length peptide (Zaretsky and Zaretskaia 2020). Therefore, the main function makes lysosomes a primary suspect in the initiation of the biochemical pathway leading to cellular death. Also, the last, but as will be explained below not the least, lysosomal acidic content promotes the formation of very large membrane amyloid channels ((Lin and Kagan 2002). Therefore, this organelle first produces an amyloid fragment, and then provides perfect conditions for the incorporation of channel-forming units and the formation of membrane channels.

### Amyloid Channels can be Giant (Most are Just Large)

The most dramatic feature of amyloid channels is their conductance—the absolute numbers of conductance are at least two orders larger than typical sodium channels (Arispe, Pollard et al. 1993). The conductance is a reflection of the pore size. It is reasonable that due to an extremely large pore, the channel is not selective because various molecules can fit inside and pass through a membrane. Amyloid channels are made of multiple copies of peptide that form a barrel-like structure (Durell, Guy et al. 1994), which is similar to the voltage-dependent anion channel (VDAC) of the outer mitochondrial membrane (Colombini 2012). The conductance of VDAC channel is in the same nanosiemens range as the amyloid channel (Arispe, Pollard et al. 1993, Lin and Kagan 2002, Micelli, Meleleo et al. 2004, Bode, Baker et al. 2017). VDAC has a diameter of 2.5 nm and allows macromolecules of up to 4-6 kDa to pass (Benz 1985, Nelson and Kabir 1986, Colombini 2012). The size exclusion of VDAC is determined by the complex shape of the pore, which is not a cylinder with the mentioned diameter. Unlike VDAC, which has voltage-dependence and functional selectivity (Colombini 2012), amyloid channels are less selective. Therefore, the size of the pore can have a more direct correlation with the size exclusion. However, not all channels are created equal. It is reasonable to hypothesize that the number of individual peptide molecules involved into the channel creation could be different, so pores size are also different. In fact, there is a distribution of conductances, with most of channels being relatively small, while giant conductances being rare. It was demonstrated that channels formed by fragment Aβ₂₅₋₃₅ have conductance up to 1 nS, but 90+% of channels created at neutral pH are small (below 200 pS, (Lin and Kagan 2002)).

To look on channel conductances in the context of molecular weight cut-off, we calculated molecular weights of imaginary compounds of spherical shape which have the density of globular proteins and can come through circular pores of various diameters (Erickson 2009). The diameters of the channels in non-transmissible membrane were calculated to correspond the pores filled with saline (Bode, Baker et al. 2017). The graph of MWCO vs conductance for these ideal conditions is shown overlayed over the reproduction of experimental data on the conductivity of channels. In these ideal conditions, passage of 50 kDa protein requires only 2.4 nm pore with conductance of 2.2 nS. In experiments performed at neutral pH, most pores are small and did not allow leakage of calcium-sensitive probe Fluo-3 (Zaretsky and Zaretskaia 2020). Most likely, at pH 7.4 membranes will remain impermeable to smaller Lucifer Yellow. However, lysosomal content is acidic (pH lower than 5, (Mindell 2012)). In acidic conditions, the channels formed by amyloid fragment are larger and ˜30% of them allow for the passage of LY. Channel-forming mechanism of lysosomal permeabilization readily explain why endocytosed fluorescently-labeled amyloid is not transported into the cytoplasm and remain colabelled with lysosomes creating a punctate pattern of fluorescence (Wesen, Jeffries et al. 2017): oligomers of 4 kDa molecules can pass only extremely large channels, but there are very few of them to pass enough of label to register microscopically. Nevertheless, it looks like extremely large channels are formed because the leakage of large proteins, such as β-hexoseaminidase (MW 150 kDa) is increased after exposure to Aβ (Ji, Miranda et al. 2002).

### Is Channel-Forming Activity Biologically Significant?

An observation of single channels in electrophysiological experiments described by Arispe and others suggests that the channels are formed in low numbers. This contradicts the fact that the peptide can be toxic in cellular cultures containing millions of cells. A significant decrease of cell viability corresponds better to experimental data obtained in liposomes, where the effects of Aβ were comparable to the effects of ionophores (Alarcon, Brito et al. 2006). On the other hand, if Aβ actually permeabilizes all liposomes in the suspension, as can be deduced from the data of Alarcon et al (Alarcon, Brito et al. 2006), why don't all cells die quickly in amyloid toxicity assays employing a high concentration of peptide?

To improve the quantitative aspect of studying the permeabilization of liposomes by A3, we developed a flow cytometric technique which allowed us to estimate the number of channel-forming units in the solutions of peptide (Zaretsky and Zaretskaia 2020). This number appears extremely small compared to the total amount of peptide. In model conditions, one channel is formed out of approximately 10¹² molecules of Aβ₂₅₋₃₅. The low ratio of channels created by the peptide would not allow for the studying of channel formation using typical methods of protein biology, such as spectroscopy or NMR, for the reason that the signal of interest being too low. Even though the proportion of channels is small, when the peptide was added in micromolar concentrations, thousands of permeabilized vesicles could be observed, which makes the power of channel formation biologically relevant.

To conclude, biophysical studies demonstrate that short amyloid fragments form non-selective membrane ion channels in negatively charged membranes. This phenomenon can be sufficiently powerful to be the mechanism of cellular toxicity. Membrane damage by low concentrations of Aβ is more likely to be linked to short fragments than to full-length peptides.

### Limitations of the Amyloid Channel Hypothesis

Amyloid channels readily explain the primary damage to live cells—exogenous Aβ in cell cultures or intercellular Aβ in tissues would induce serious ion disturbance across plasma membranes, which can be fatal for any cell and be disruptive for the function of neurons that are still alive. However, there are multiple significant problems undermining the value of the amyloid channel theory.

Starting with the first publications, major attention was attracted to calcium homeostasis changes induced by Aβ channels. The permeabilization of membranes to calcium after exposure to Aβ was described at both the membrane and cellular levels (Arispe, Rojas et al. 1993, Arispe, Pollard et al. 1994, Arispe, Pollard et al. 1994, Abramov, Canevari et al. 2004, Alarcon, Brito et al. 2006, Lin and Arispe 2015, Drews, Flint et al. 2016). It is not difficult to link the permeabilization of the plasma membrane to calcium with mitochondrial damage and cell death. An increase of intracellular calcium would require a removal of calcium to preserve the normal function of cell. Mitochondria are one of the reservoirs for calcium excess. If calcium is transferred to the mitochondria for a prolonged time, a calcium overload activates various pathological processes, such as a mitochondrial membrane permeability transition, which in turn leads to apoptosis and necrosis (Rizzuto, De Stefani et al. 2012). However, the channels are not selective and pass various cations, so such selective attention to calcium is not justified due to the extreme conductance of channels. The permeability to calcium in various studies was only 1.3-3.8 times higher than to sodium (Arispe, Rojas et al. 1993, Mirzabekov, Lin et al. 1994). The changes of intracellular concentrations of major cations induced by exposure to Aβ mirror each other—once the cellular membrane opens for one ion, others start flowing across, too (Abramov, Canevari et al. 2004).

The ion disturbance induced by the channel formation in the plasma membrane, which can be predicted from conductance data, is much stronger than observed biological effects. In all studies, the effects of peptides on membranes in model systems set in quickly (immediately or within several minutes at most). Based on channel conductance, even a single channel would completely dissipate the ion gradient in a cell of any reasonable size on a time scale of minutes (Arispe, Pollard et al. 1993). The dissipation of membrane ion gradients would also mean the dissipation of resting membrane potential. Surprisingly, to our knowledge, there are no published observations of such a phenomenon. Increased neuronal activity in various models of AD (Parodi, Sepulveda et al. 2010, Busche, Chen et al. 2012, Busche and Konnerth 2016) could be interpreted as a consequence of mild membrane depolarization, but calculations predict a complete depolarization incompatible with an ability to generate action potentials.

Next, the reason why negative charge of the membrane is required for the channel formation, if it is formed by a full-length peptide, was never properly explained. Both Aβ₁₋₄₀ and Aβ₁₋₄₂ carry a negative charge at a neutral pH, which contradicts the need for electrostatic interaction. In contrast, the short fragment Aβ₂₅₋₃₅ carries a positive charge at any pH below 10. Electrostatic interactions would readily explain why C-terminal fragments interact with a negatively charged membrane, but not with a neutral membrane. If amyloid channels are involved in AD pathogenesis, then, most likely, it is fragments that form the channels, not full-length peptides.

Most importantly, the damage to plasma membranes does not explain a well-known phenomenon associated with AD: autophagy failure. The dramatically increased presence of autophagic vacuoles is one of the features of Alzheimer's disease (Nixon, Wegiel et al. 2005). While mitochondrial dysfunction—another hallmark of AD (Blass 2000, Chen and Yan 2007, Chen and Yan 2010, Eckert, Schmitt et al. 2011, Demetrius, Magistretti et al. 2015)—can be linked to ion disturbances induced by membrane permeabilization, typical implementation of amyloid membrane channel theory does not explain the link between lysosomal dysfunction and membrane channel formation.

### Amyloid Degradation Toxicity Hypothesis of AD Pathogenesis

This integrative hypothesis was proposed by us (Zaretsky and Zaretskaia 2020, Zaretsky and Zaretskaia 2021). It builds on the amyloid channel theory and aims to explain major hallmarks of AD such as intracellular accumulation of amyloid, decreased brain metabolism, and defects of autophagy. This hypothesis also allows for an interpretation of the delay before cellular responses are observed after exposure to Aβ and why cell death occurs multiple hours after exposure.

The hypothesis can be summarized as a sequence of molecular events (FIGS. 8 and 9). Aβ is taken through endocytosis. After merging of endocytic vesicle with a lysosome, the peptide is degraded. In normally functioning cells, short fragments generated by endopeptidases do not accumulate, but are degraded by exopeptidases which are most active in acidic environment. If some fragments aggregate, they can form membrane channels which are not ion-specific and can allow to pass relatively large compounds. Channel formation explains lysosomal permeabilization. Among various ions, which become equilibrated between the interior of the lysosome and the cytosol, protons are most critical for lysosomal function. Even a single channel allows the dissipation of proton gradient across a membrane of a particular organelle. In neutral environment, most lysosomal proteases are inactive.

The appearance of dysfunctional lysosome is the main consequence of lysosomal permeabilization. After lysosomal damage, there are several potential pathways leading to cell death. Most obvious one is the leakage of lysosomal enzymes through permeabilized membranes (2, FIG. 8). Enzymes either directly digest cellular content (necrosis) or activate cytoplasmic caspases to induce apoptotic cell death. Second pathway could involve leakage of channel-forming peptides (1, FIG. 8). Likely target for leaked fragments is the internal leaflet of plasma membrane. Activation of these two pathways leads to a relatively fast demise of a cell.

In contrast, third pathway (3, FIG. 8) involves a prolonged accumulation of multiple failed lysosomes, because their cargo is not digested. Taken amyloid is not processed, therefore, it accumulates. Increasing the number of failed lysosomes prevents the recycling of other failing organelles, with mitochondria being the most sensitive to the loss of cellular quality control. Failed mitochondria produce toxic ROS (well-known feature of AD), but suppressed mitogenesis results in a hypometabolism typical for AD.

Within this concept, the etiology of the disease is the formation of beta-amyloid fragments which form membrane channels. Stopping the production of toxic fragments means targeting the etiology of AD.

### Amyloid Degradation Hypothesis Defines Novel Pharmacological Targets

Blocking channels allowed to inhibit amyloid toxicity in some in vitro studies (Chafekar, Baas et al. 2008, Dominguez-Prieto, Velasco et al. 2018). However, while smaller channels (<400 pS) can be effectively blocked by tromethamine, the same blocker was less effective in super-high conductance giant channels (>1 nS): it is not surprising that blocking large hole is more difficult than a small one. Considering that slow development of the disease could be linked to a rarity of the appearance of giant channels, because cell death is most likely associated with a leakage of lysosomal enzymes through super-large channels in lysosomal membranes, we can expect that attempts to block channels directly could fail. Also, due to giant conductance, if channels are already formed, they need to be blocked essentially completely, but electrophysiological measurements show significant remaining currents after the blockade by small molecules (Arispe, Rojas et al. 1993, Arispe, Pollard et al. 1996, Arispe, Diaz et al. 2007). The treatment would be much more effective if such channels are not formed in the very first place. Considering that channels are formed from the fragments, this can be accomplished by a selective inhibition of proteases cleaving amyloid into toxic fragments. There are multiple lysosomal proteases, which are able to digest amyloid. Some of them are endoproteases, cutting long peptides in pieces, while others are exoproteases which are responsible for the degradation of fragments into amino acids. By inhibiting only those proteases which are responsible for the formation of channel-forming fragments, it is possible to suppress the channel formation without significant inhibition of overall amyloid metabolism.

Until recently, the amyloid plaques were considered as etiological for AD, so anything that can increase the density of plaques was considered as a factor promoting the progression of AD. Therefore, protease inhibitors were studied in the context of AD as an aggravating factor and the data were published. Screening peer-reviewed publications, we found independently collected and peer-reviewed evidence that inhibitors of proteolytic enzymes can protect against amyloid toxicity. Therefore, despite no direct studies aimed to test if inhibition of proteolytic enzymes can protect against AD and this hypothesis was never proposed, the data supporting our claim exists in peer-reviewed studies.

Example 1 (FIG. 14, adapted from (Frautschy, Horn et al. 1998)). Chronic infusion of Aβ increases the expression of apoptotic marker TUNEL in the brain. The increased expression by itself is in line with our hypothesis that leaking lysosomal enzymes can activate apoptotic mechanisms. Non-selective inhibition of proteolytic activity with simultaneous i.c.v. infusion of leupeptin promoted apoptosis and an accumulation of extracellular and intracellular Aβ immunoreactivity. In contrast, more selective serine protease inhibitor aprotinin prevented Aβ-induced apoptosis and did not exacerbate intracellular accumulation of amyloid. The difference between leupeptin and aprotinin is in the selectivity of protease inhibition: aprotinin is serine protease inhibitor, while leupeptin has much wider range of targets including both serine and cysteine proteases. Cysteine proteases include cathepsins which have high exopeptidase activity and are degrading fragments into amino acids, therefore the treatment with leupeptin results in accumulation of fragments rather than prevention of their formation.

Example 2. (FIG. 15 adapted from (Schubert 1997)). Natural serpins (serine peptidase inhibitors) such as α1-anti-chymotrypsin (ACT) can protect against Aβ-induced drop in cell viability but have a bell-shaped dose-dependence of protection. The optimal dose for inhibition of amyloid toxicity was similar in tests with different concentrations of Aβ but depended on the cell strain: in 15 cells it was 10⁻⁷M, in primary cortical cultures—10⁻⁸M. Bell-shaped curve can explain contradicting data in other studies—ACT appeared not effective in (Ma, Brewer et al. 1996) but attenuated Aβ₁₋₄₂ toxicity without affecting fibril formation in (Aksenov, Aksenova et al. 1996). Similarly, cystatin C (endogenous cysteine protease inhibitor) prevented the drop of cell survival induced by Aβ₁₋₄₂ (Tizon, Ribe et al. 2010). Bell-shaped dose-dependence reflects the inhibitory action of anti-chymotrypsin on various proteases—chymotrypsin is a serine protease, so naturally present antagonist (ACT) has highest affinity to them, and at lowest concentrations mostly prevents the formation of channel-forming fragments. In contrast, in higher concentrations, ACT becomes non-selective and inhibits a greater range of proteases (including cysteine proteases) and thus prevents the degradation of such fragments, promoting Aβ toxicity.

To conclude, the inhibition of proteases allows to prevent amyloid toxicity without a significant effect on the overall metabolism of beta-amyloid.

Late-Onset Alzheimer's Disease is Associated with Increased Cellular Uptake of Beta-Amyloid

By processing data on two major biomarkers of Alzheimer's disease (concentration of beta-amyloid in the CSF and the density of amyloid deposits in the brain measured by PET using appropriate FDA-approved test substances), we estimated the intensity of cellular beta-amyloid uptake.

In general, two parameters have strong negative correlation (FIG. 1A). However, even after considering amyloid deposition density, higher levels of beta-amyloid in the CSF are associated with better clinical outcome (FIG. 1B). To interpret the data, we estimated parameters of beta-amyloid turnover in the brain of patients using mathematical model shown at the FIG. 2. While we found that the synthesis of beta-amyloid is not different between AD patients and subjects with normal cognition, the aggregation-independent elimination of beta-amyloid from interstitial fluid (parameter KF in the model) is higher in AD patients. Considering that elimination through the cerebrospinal fluid is not increased in AD patients, the increase of KF can be explained only by dramatic increase in cellular uptake of beta-amyloid (FIG. 3E).

Increased cellular uptake in AD patients matches the concept of increased production of toxic (channel-forming) amyloid fragments as a potential mechanism of AD.

### Relevance of Amyloid Degradation Toxicity to Other (Neuro)Degenerative Diseases

Different neurodegenerative disease, such as Alzheimer's disease, ALS etc have surprisingly similar phenomenology: it includes neuronal death associated with decreased neural metabolism and lysosomal failure. While the etiologies of diseases are associated with different proteins, all these proteins are known to have beta-sheets as a secondary structure underlying protein aggregation. Many diseases are associated with tissue accumulation of aggregated proteins which appears as amorphous clumps of protein called amyloid. The structure of beta-sheet-forming peptide sequences inside these proteins contains portions which carry features typical for membrane channel formation: along with the ability to form beta-sheets, these sequences are mostly constructed by lipophilic amino acids, while carrying at least one positive amino acid such as lysine or arginine. We claim that similar biochemical mechanisms are involved in the pathophysiology of various neurodegenerative diseases, specifically, the degradation of involved proteins by proteolytic enzymes results in the appearance of peptide fragments which are able to form membrane channels. Permeabilization of membranes by these channels initiates biochemical processes leading to the cell death. Therefore, the prevention of proteolytic degradation in the way which decreases the formation of toxic forms without significantly affecting total catabolism of said peptides is the way to treat these neurodegenerative diseases. Similarly to what was shown in studies using beta-amyloid, inhibitors of proteases (such as anti-chymotrypsin) prevent amylin-induced cellular toxicity in pancreatic islet cell tumor line (FIG. 17).

### SUMMARY OF INVENTION

In this invention we describe the method to treat amyloid diseases by preventing the formation of peptide fragments which can form membrane channels. Inhibition of amyloid membrane channel formation prevents permeabilization of lysosomal and other cellular membranes induced by amyloid peptides. If such permeabilization is not prevented, leaking lysosomal enzymes activate cellular necrosis and apoptosis, while dysfunctional lysosomes do not perform normal cellular repair which results in the accumulation of damaged mitochondria producing toxic products. If amyloid fragments reach other membranes, they can permeabilize them and affect intracellular electrolyte balance (needed for neuronal function) and induce calcium overload (activates apoptosis leading to cell death). Therefore, prevention of the accumulation of toxic fragments during amyloid digestion eventually prevents or slows down cell death induced by said proteins. Various embodiments of present invention provide the methods of high-throughput testing of compounds for potential medical use to treat misfolding proteins-caused diseases such as Alzheimer's disease.

### BRIEF DESCRIPTION OF DRAWINGS

FIG. 1. Correlation of diagnosis of Alzheimer's disease with values of two major biomarkers of AD.

The data on concentrations of soluble Aβ42 in cerebrospinal fluid (CSF-Aβ42) and the density of amyloid depositions in the brain measured by PET using appropriate ¹⁸F-conjugated label were obtained from the Alzheimer's Disease Neuroimaging Initiative (ADNI) database (adni.loni.usc.edu). The investigators within the ADNI contributed to the design and implementation of ADNI and/or provided data but did not participate in analysis or making any conclusions relevant to this invention. A complete listing of ADNI investigators can be found at: http://adni.loni.usc.edu/wp-content/uploads/how to_apply/ADNI_Acknowledgement_List.pdf
A. Scatter plot of CSF-Aβ42 (in ng/ml) vs beta-amyloid load (in centiloids) in patients with Alzheimer's disease (AD) and subject with normal cognition (NC). Most patients with AD have significant amyloid accumulation (located to the right of the line at 50 CL), while data points for most patients with normal cognition are located to the left of the line at 20 CL (no noticeable accumulation of amyloid depositions in the brain). However, some AD patients have low levels of amyloid depositions, while some subjects with normal cognition have significant accumulation of amyloid depositions.
B. Due a strong negative correlation with CSF-Aβ42, depositions of amyloid in the brain are frequently considered interchangeable methods of AD diagnostics. However, CSF-Aβ42 provide independent diagnostic information. Within a subgroup with high or low amyloid load, the level of soluble beta-amyloid in the CSF correlates with the probability of AD diagnosis: the subjects with high level of soluble beta-amyloid are almost free of disease, while subjects with low levels are prone to the development of AD.

Such distribution of two biomarkers is defined by dramatically increased cellular uptake of soluble beta-amyloid (the analysis is shown at the FIGS. 2&3), which is the molecular mechanism of Alzheimer's disease according to recently proposed by us amyloid degradation toxicity hypothesis.

FIG. 2. A schematic of the single compartment model of beta-amyloid turnover used to describe the mathematical relationship between CSF-Aβ42 and amyloid load in the brain.

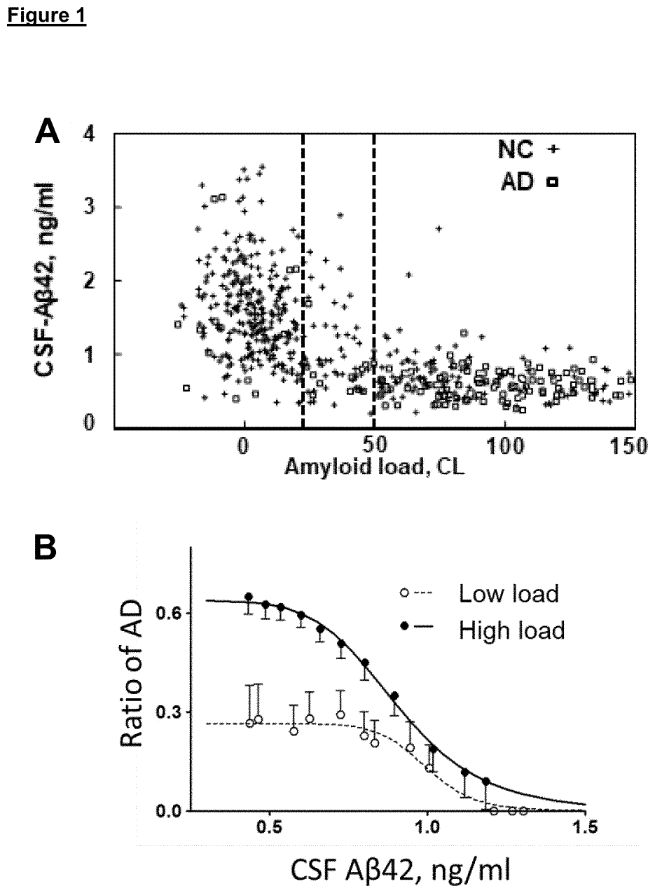

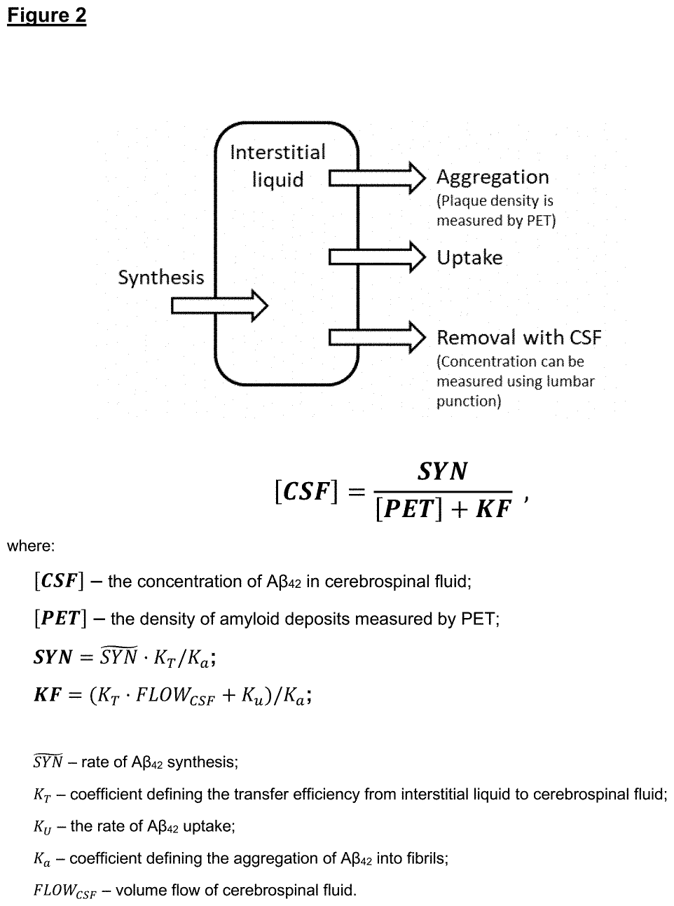

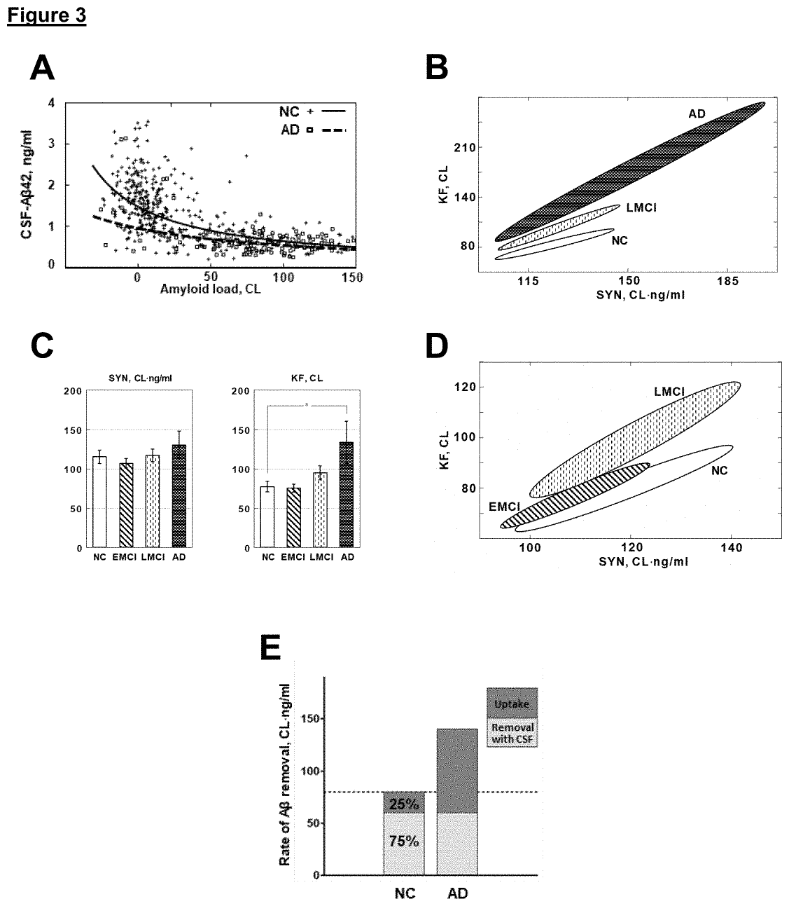

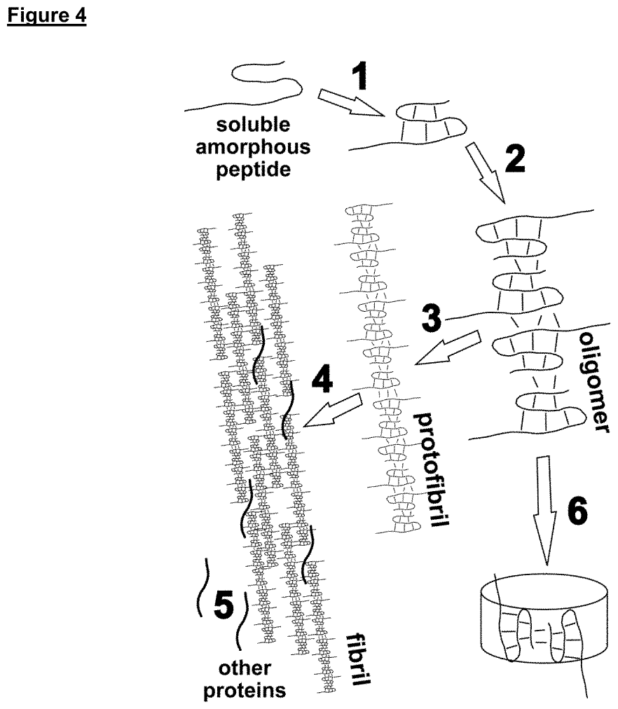

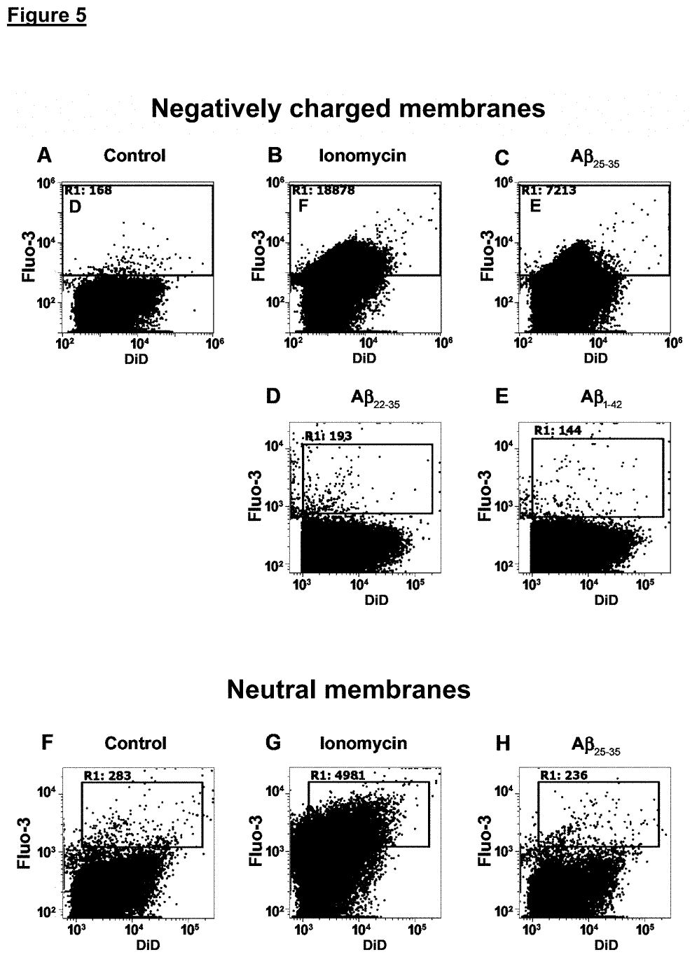

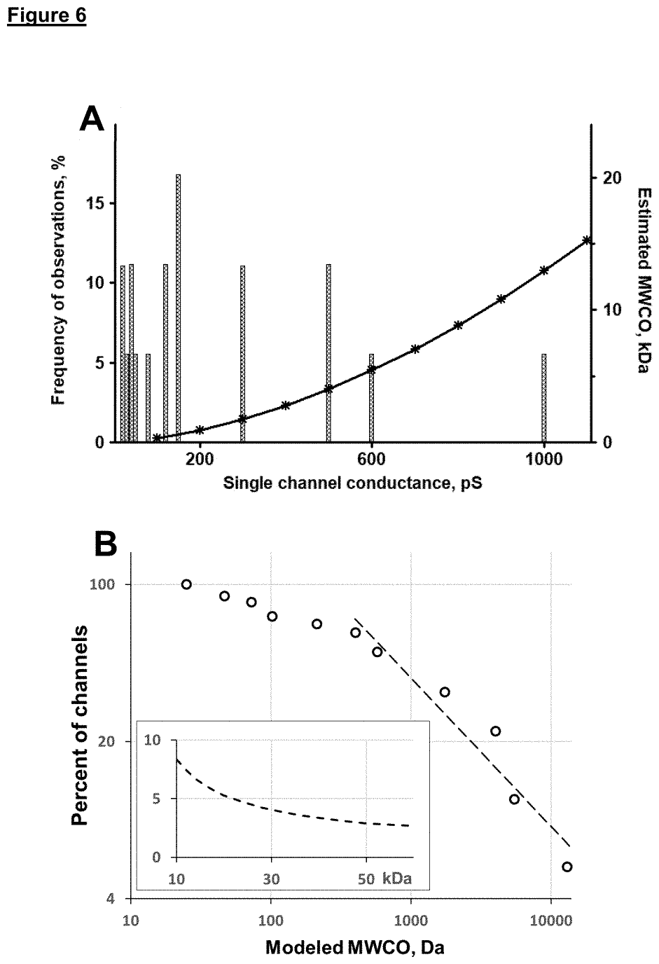

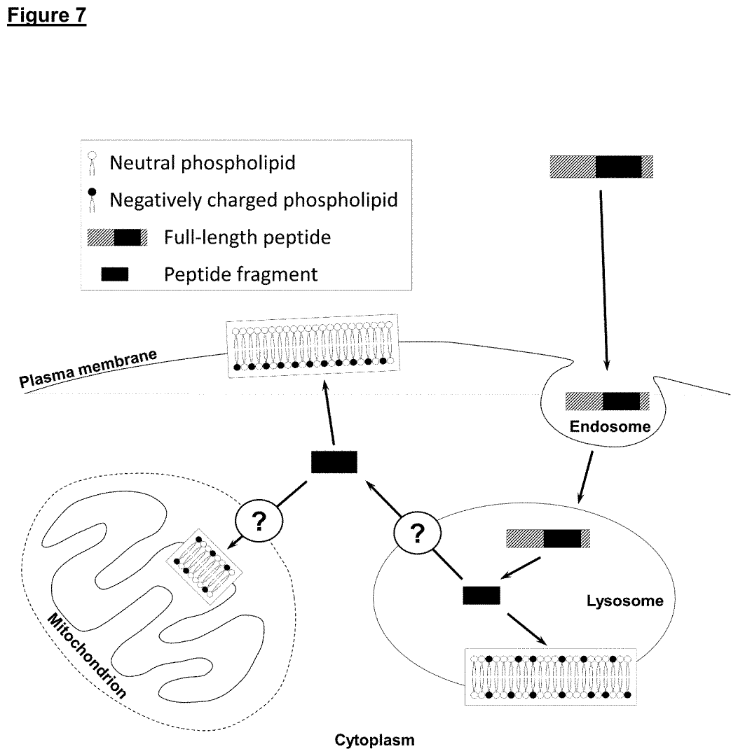

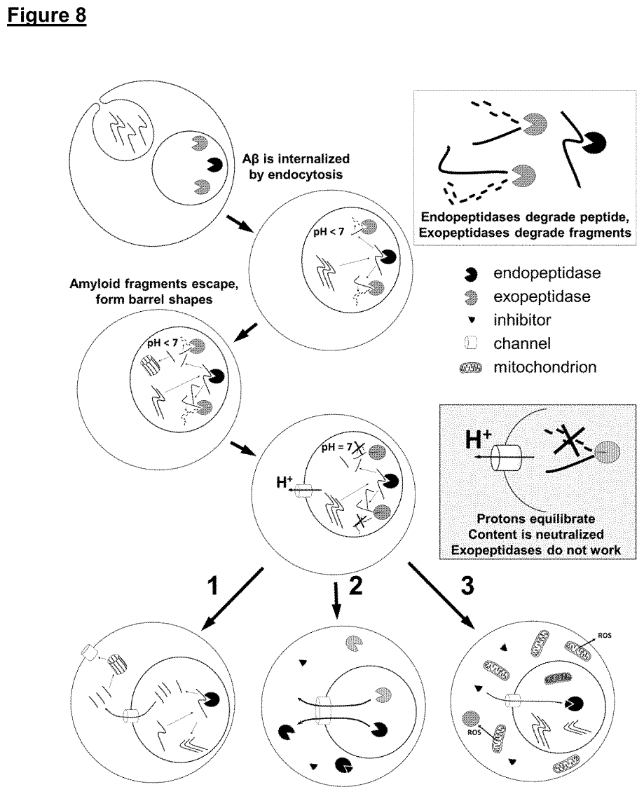

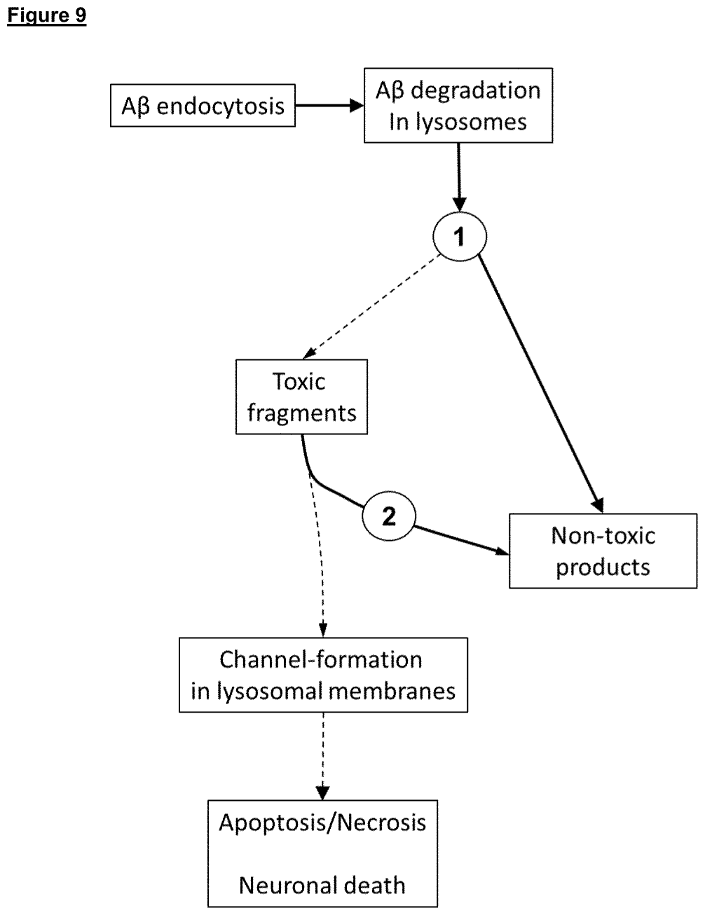

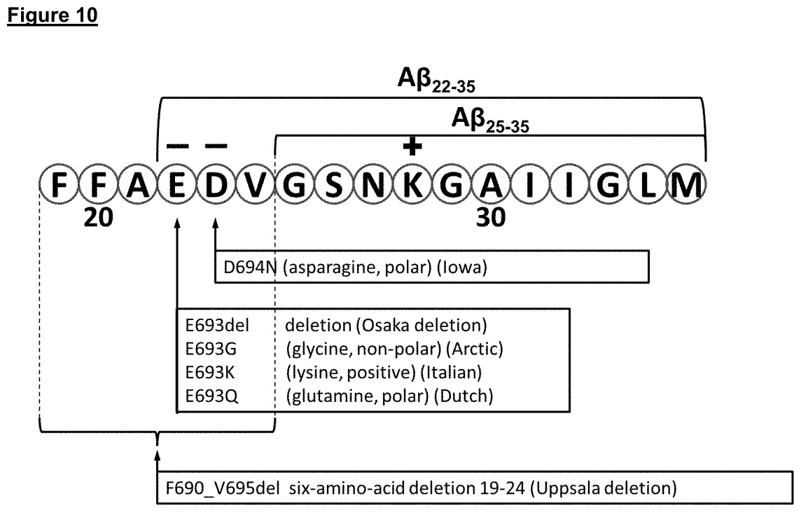

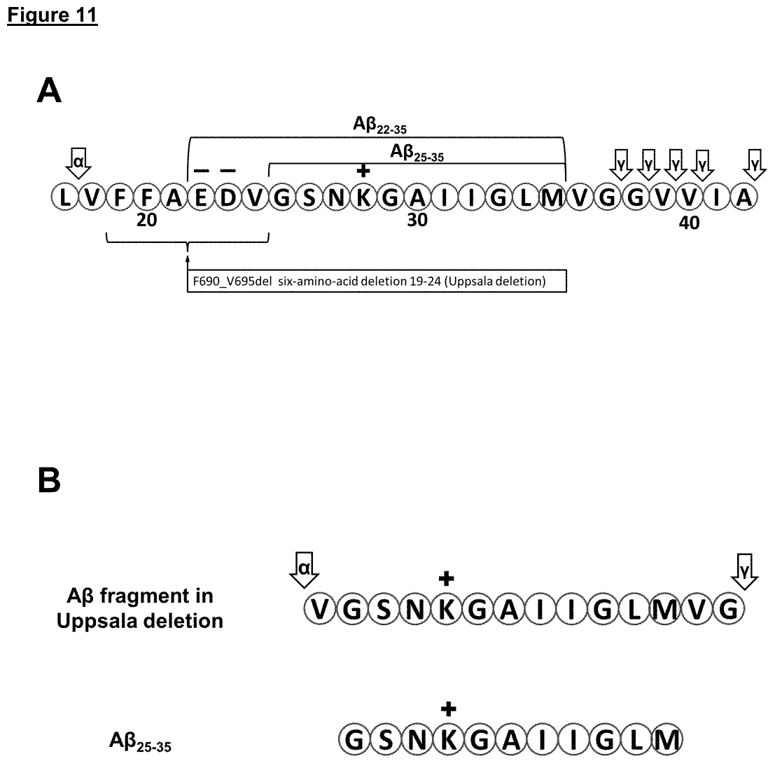

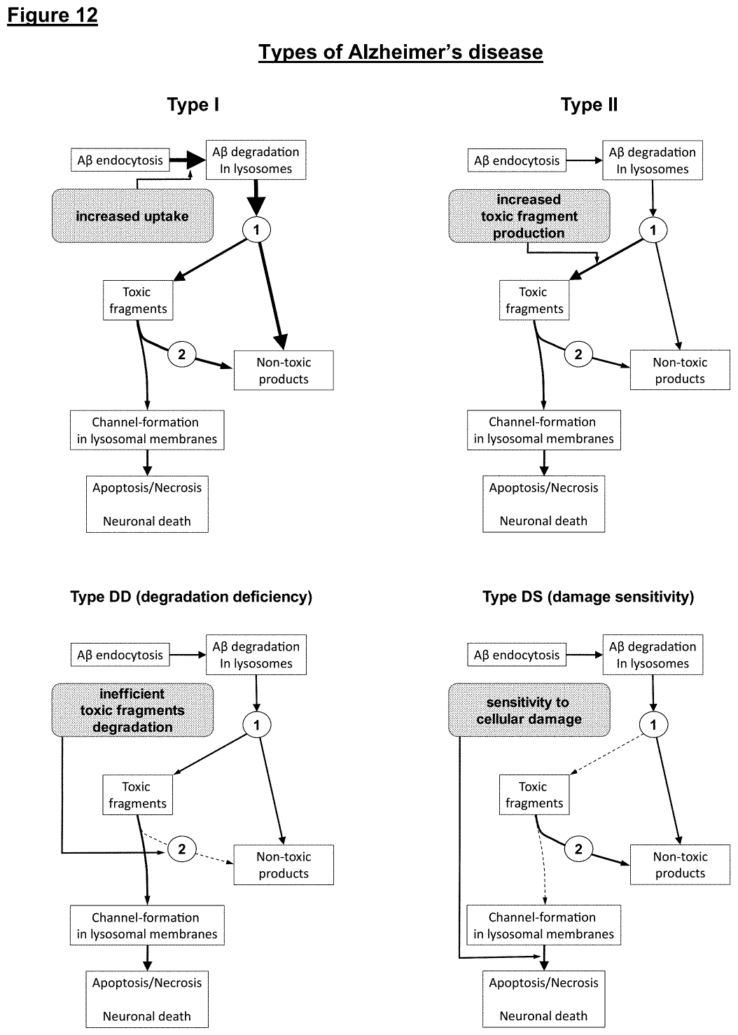

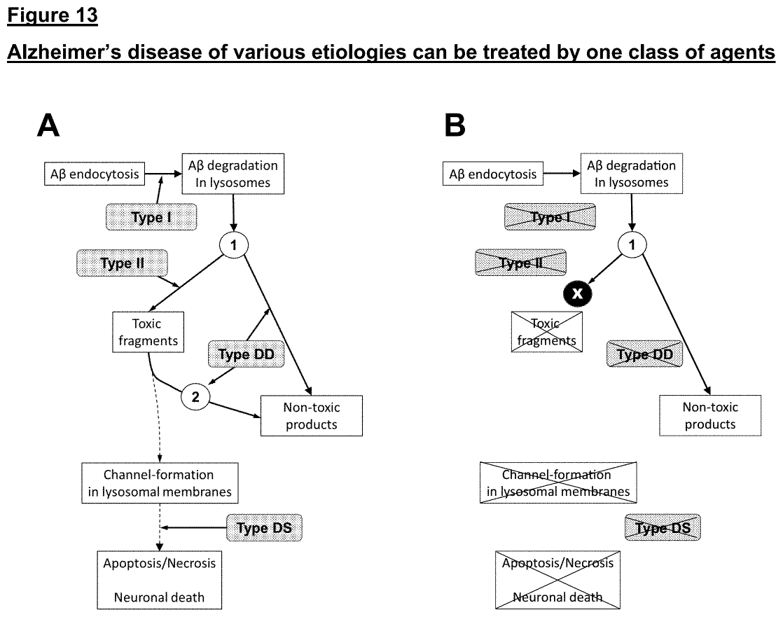

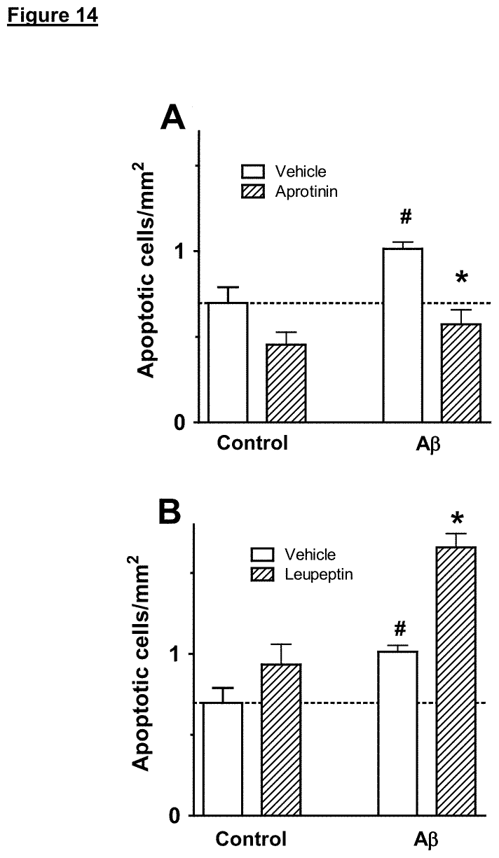

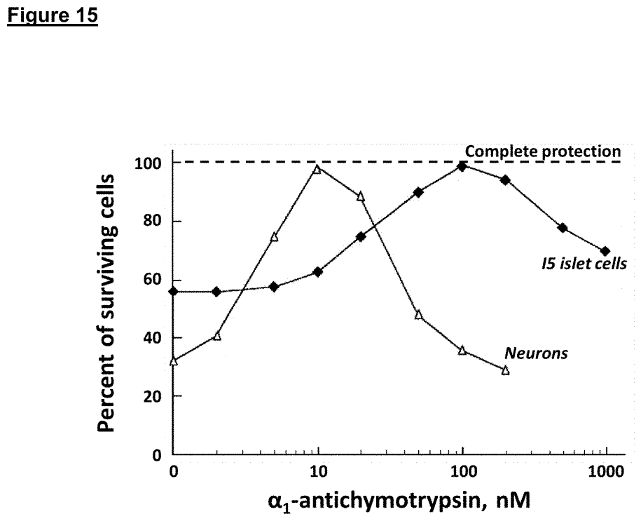

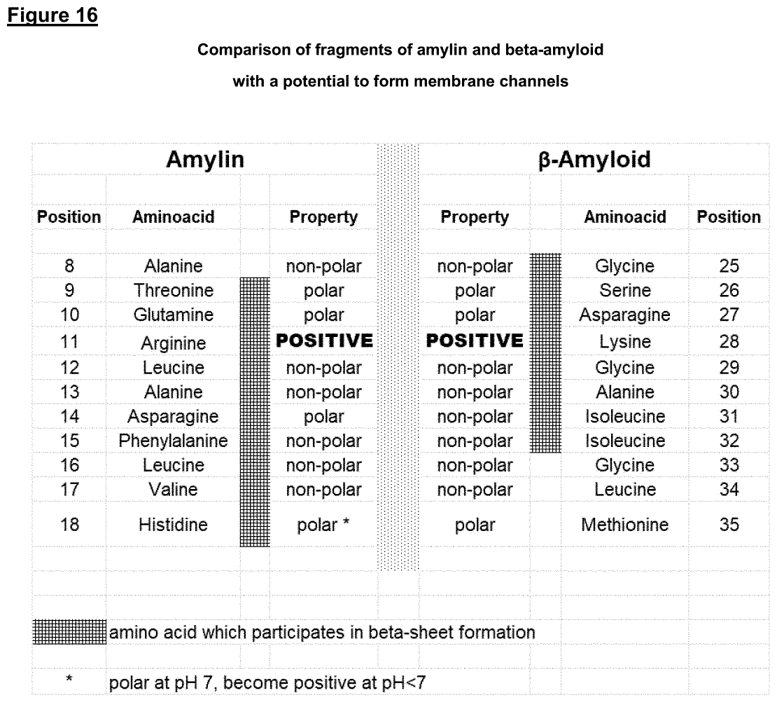

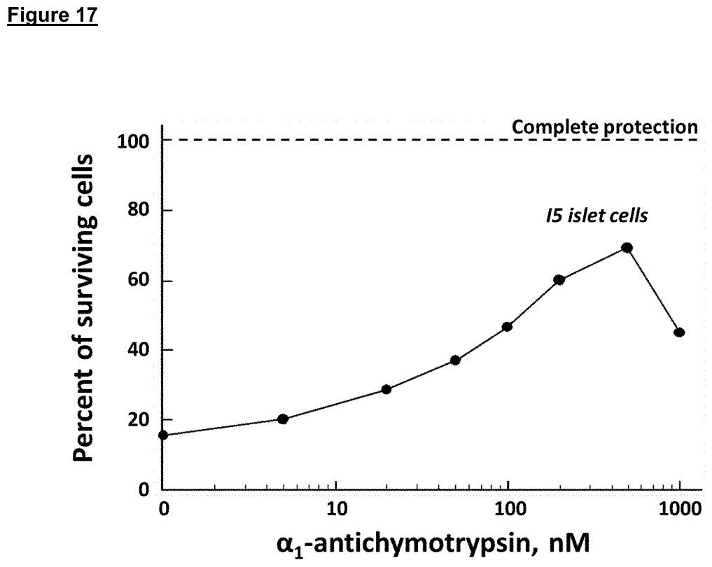

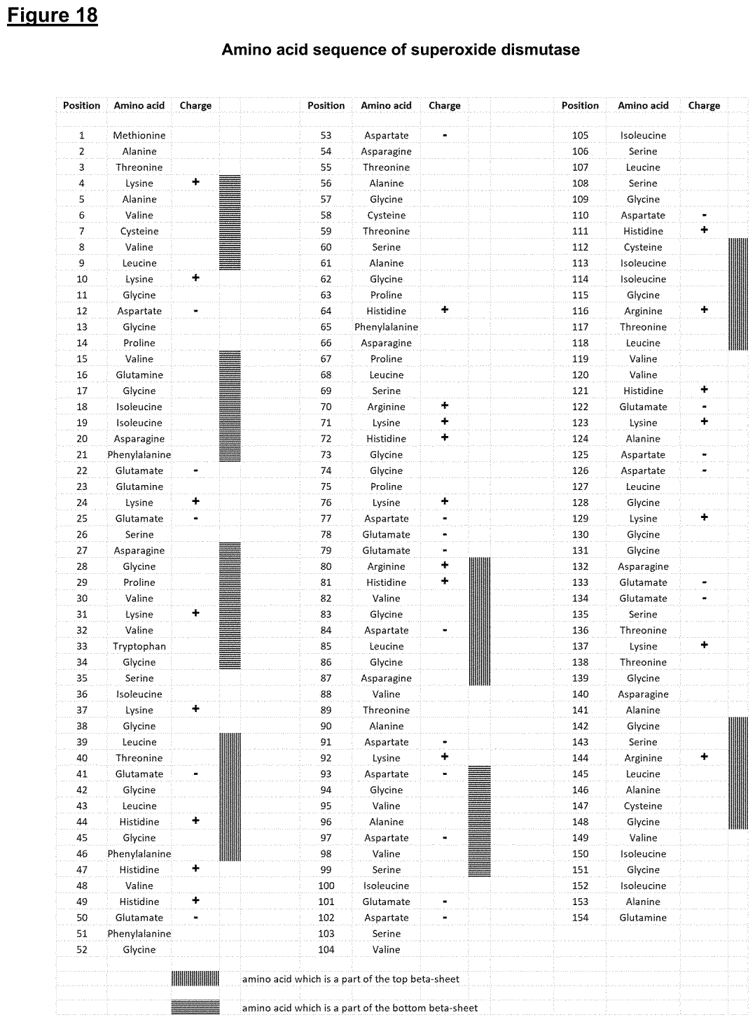

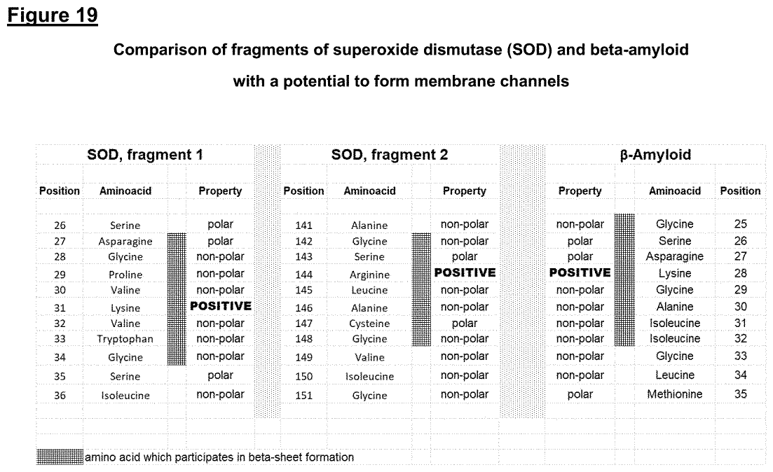

To analyze the data shown at the FIG. 1A, we used mathematical model describing the schematic shown at this Figure. The concentration of soluble amyloid in the interstitial fluid (ISF, the liquid between cells and neurons in the brain), which we denote as [ISF], is defined by several processes: 1) synthesis by cells, 2) filtration of the protein into the CSF, 3) aggregation into non-soluble plaques, and 4) uptake by cells. The model is based on several assumptions:

Synthesis rate [symbol] (SYN) is independent of both interstitial Aβ42 and the density of plaques.

The rate of removal of the protein through the CSF is a product of the CSF removal rate (FLOW_{CSF}) and CSF-Aβ42 ([CSF]): FLOW_{CSF}·[CSF].

The concentrations of the soluble beta-amyloid in the ISF and the CSF have a similar order of magnitude and are correlated. The model assumes a linear relationship between the concentrations of soluble Aβ42 in the ISF and the CSF with a coefficient of transfer K_{T}: [CSF]=K_{T}·[ISF].

Existing plaques serve as seeds for aggregation of soluble Aβ42 in the ISF. The rate of loss of soluble Aβ42 in the ISF due to aggregation is the product Aβ42 concentration in the ISF, the concentration of plaques ([PET], calculated from the intensity of the PET signal), and the coefficient of aggregation Kₐ: Kₐ·[PET]·[ISF].

The rate of cellular uptake of soluble Aβ42 is proportional to the interstitial concentration [ISF] with a coefficient of uptake Kᵤ: Kᵤ·[ISF].

FIG. 3. Beta-amyloid turnover parameters in subjects with normal cognition (NC), patients with Alzheimer's disease (AD), patients with late-onset mild cognitive impairment (LMCI) and early-onset mild cognitive impairment (EMCI).

The parameters were inferred from two major AD biomarkers (CSF-Aβ42 and beta-amyloid density) in research subjects from the ADNI database. The data on concentrations of soluble Aβ42 in cerebrospinal fluid (CSF-Aβ42) and the density of amyloid depositions in the brain measured by PET using appropriate 18F-conjugated label were obtained from the Alzheimer's Disease Neuroimaging Initiative (ADNI) database (adni.loni.usc.edu). The investigators within the ADNI contributed to the design and implementation of ADNI and/or provided data but did not participate in analysis or making any conclusions relevant to this invention. A complete listing of ADNI investigators can be found at: http://adni.loni.usc.edu/wp-content/uploads/how to_apply/ADNI_Acknowledgement_List.pdf
A. Scatter plot of CSF-Aβ42 vs beta-amyloid load for subjects with NC and AD patients showing the lines representing best fits by Equation (FIG. 2) for each group.
B. The 95% confidence regions of the parameters characterizing beta-amyloid turnover in the three groups (NC, AD, LMCI). All three groups are different if both KF and SYN are considered.
C. A comparison of beta-amyloid turnover parameters in subjects with normal cognition (NC), patients with either early-onset and late-onset mild cognitive impairment (EMCI and LMCI), and patients with Alzheimer's disease (AD). The inferred values of the beta-amyloid synthesis rate (SYN) and the removal rate (KF) for all studied groups. * Only the values of KF for the NC and AD groups are statistically different (z-test, p<0.05).
D. The 95% confidence regions of the parameters characterizing beta-amyloid turnover in the NC, EMCI, and LMCI groups. The confidence regions for the NC and LMCI groups do not overlap, while the confidence regions for the NC and EMCI groups do.
E. The difference in the aggregation-independent amyloid removal rate (parameter KF) between NC subjects and AD patients translates into a much greater relative difference in cellular amyloid uptake rate. CSF flow in AD patients is either the same or lower than in NC subjects while the removal with the CSF is a prevalent mechanism defining interstitial concentration of soluble amyloid. The dark-gray and light-gray parts of the bars represent the cellular amyloid uptake rate and the rate of removal through the CSF (the components of the rate of amyloid removal), respectively. If the ratio of the two components is 50/50 in the NC group, the cellular uptake rate is 2.5 times greater in the AD group than in the NC group. If the ratio is 75/25, the difference is 4-fold.

FIG. 4. The schematic of polymerization of amyloidogenic proteins.

Amyloid peptides are initially soluble without secondary or tertiary structure. With time, they are stabilized by intra- and intermolecular hydrogen bonds (1 and 2, correspondingly) forming beta-pleated sheets (one of major secondary structures in proteins). Elongation of these supramolecular structures results in formation of protofibrils which have 3-sheet core with polypeptide tails looking to the sides of the protofibril (3). Protofibrils stick to each other through interaction between side polypeptide chains (4) and may involve other proteins (5), which may or may not be containing carbohydrate and lipid components (glyco- and lipoproteins). At oligomeric stage, beta-sheet can form barrel-like structures (6), which can incorporate into lipid membranes and serve as ion channels.

FIG. 5. Aβ₂₅₋₃₅, unlike Aβ₂₂₋₃₅ and Aβ₁₋₄₂, makes negatively charged liposomes permeable to calcium.

The figure is a composite of data which was presented and analyzed in detail in (Zaretsky and Zaretskaia 2020, Zaretsky and Zaretskaia 2020). Liposomes (400 nm) were made of either phosphatidylserine (A-E, negatively charged lipid) or phosphatidylcholine (F-H, neutral lipid) with added DiD and were extruded in the Ca-free buffer containing 1 mM Fluo-3. After the addition of calcium, aliquots of peptide were added. Ionomycin was used as a positive control. Fluorescence of liposomes was analyzed using a flow cytometer. Each dot at the graph represent a single liposome registered in the flow. The intensity of DiD fluorescence characterizes the amount of membrane material in the liposome, while the fluorescence of Fluo-3 depends on the intraliposomal presence of calcium. Permeabilized liposomes (Fluo-3 is saturated with calcium entering through damaged membrane) appear at the graph above the rest of liposomes. The liposomes inside rectangle (area R1) are permeabilized, and the number of liposomes which were counted to be within the area is shown. Total number of analyzed events represented at each graph is approximately 100,000.
A. Control phosphatidylserine liposomes (negatively charged) are not permeable to calcium; therefore, the number of permeable liposomes is low.
B. The addition of ionomycin permeabilizes liposomes, so thousands of liposomes become permeable to calcium.
C. The addition of Aβ₂₅₋₃₅ which carries positive charge creates multiple liposomes permeable to calcium. This number depends on the concentration of added peptide (see the data in the (Zaretsky and Zaretskaia 2020).
D. Longer peptide Aβ₂₂₋₃₅ which carries overall negative charge does not permeabilize liposomes.
E. Full-length amyloid peptide Aβ₁₋₄₂ also does not permeabilize liposomal membranes.
F. Control phosphatidylcholine liposomes (non-charged) are not permeable to calcium; similar to observations in negatively charged liposomes (Panel A), the number of permeable liposomes is low.
G. As in case of negatively charged liposomes (Panel B), ionomycin permeabilizes thousands of liposomes.
H. In contrast to negatively charged liposomes, neutral liposomes are not permeabilized by Aβ₂₅₋₃₅ (the number of permeabilized liposomes is not more than in control, Panel F).

FIG. 6. Distribution of amyloid channel conductances and of corresponding theoretically predicted molecular weight cut-offs. The Figures are adapted from (Zaretsky, Zaretskaia et al. 2021)

Amyloid channels were formed by 20 μM Aβ₂₅₋₃₅ in planar lipid bilayers at pH 5.0 (to mimic the conditions inside lysosomes). The conductances of membrane channels formed by beta-amyloid have a wide range of values, from below 100 nS to above 1 nS. Most of channels (around 90% at pH 7.4) are relatively small (below 200 pS), but at acidic pH more than 30% of channels had a conductance exceeding 200 nS. Channels with giant conductance up to 1 nS are rare.
A. To look at the channel conductance in the context of molecular weight cut-off, we calculated molecular weights of compounds of spherical shape which the density of globular proteins which can come through circular pores in lipid membrane filled with saline using formulas used to calculate conductance of pores (Bode, Baker et al. 2017) and the size of proteins (Erickson 2009). In these ideal conditions, passage of 50 kDa protein requires only 2.4 nm pore with conductance of 2.2 nS.
B. The percentage of channels with a conductance which in model conditions is sufficient to pass globular proteins of various molecular mass based on the distribution shown at the Panel A. Log-log scale was used to estimate the shape of the dependence as a power function. Linear fitting of distributions was performed for conductances which correspond to MWCO exceeding 500 Da. Insert: Extrapolation of probability of giant channels based on fitting with a power function for MW up to 50 kDa. For comparison, lysosomal cathepsins (proteases) have MW of 20-30 kDa, so there is biologically significant probability that they can pass lysosomal membranes if the membranes are permeabilized by beta-amyloid fragments.

FIG. 7. The pathway of beta-amyloid metabolism in healthy cells and potential membrane targets containing negative charge required for amyloid channel formation.

Negative charge exists in several subtypes of cellular membranes: in lysosomes, inner mitochondrial membrane, and inner leaflet of plasma membrane. There is no known mechanism for channel-forming amyloid fragments to access inner leaflet of plasma membrane and mitochondria (both are considered targets based on known pathophysiology of Alzheimer's disease), unless the fragments leak from lysosomes. Giant amyloid channels can be a mechanism for such leakage. Even if channel-forming fragments can leak to the cytoplasm, the delivery to the inner mitochondrial membrane still requires yet unknown mechanism. However, mitochondrial disfunction can be explained without such transport by assuming lysosomal disfunction as shown at the FIG. 8.

FIG. 8. Amyloid degradation toxicity hypothesis (adapted from (Zaretsky and Zaretskaia 2021)).

The endocytic vesicle containing the amyloid peptide is merged with a lysosome. Endopeptidases produce various short fragments, which are mostly degraded by acidic exopeptidases. Short fragments can form non-selective membrane channels, dissipating the pH gradient. The neutralization inhibits acidic proteases with exopeptidases being inhibited more than endopeptidases. Lysosomal failure leads to cell death through several pathways. 1. Channel-forming fragments leak to the cytoplasm through the permeabilized membrane and target other membranes, including the plasma membrane. 2. Lysosomal enzymes leak to the cytoplasm and cause necrosis or activate apoptosis. 3. Dysfunctional lysosomes accumulate, and the recycling of organelles fails. Damaged mitochondria are not recycled and produce reactive oxygen species, damaging other organelles.

FIG. 9. The sequence of events resulting in neuronal death and the progression of Alzheimer's disease as suggested by the amyloid degradation toxicity hypothesis.

Beta-amyloid which is internalized by cells through endocytosis meets lysosomal degradation enzymes after endosomes merge with lysosomes. Degradation of any protein occurs through either cutting it into large fragments by endopeptidases or by cutting mono-, di- or tri-peptides from the ends of polypeptide chain by exopeptidases (which could be appropriately named di- or tri-peptidases). It is logical that after the fragments are formed, they are further degraded by exopeptidases.

Only some beta-amyloid fragments are channel-forming (and toxic), their appearance depends on the activity of endopeptidases (circle with a digit 1). It is likely that most of formed fragments are non-toxic products. Also, toxic fragments are mostly quickly degraded into non-toxic products (by other exopeptidases and endopeptidases, circle with a digit 2). All that process occurs inside lysosomes and is promoted by intralysosomal acidic conditions (pH<5). Only minor number of toxic fragments aggregate into channel-forming units and incorporate into lysosomal membranes. If this happens, it results in lysosomal disfunction which in turn produce multiple sequela associated with Alzheimer's disease, such as mitochondrial disfunction, increased production of reactive oxygen species, low brain metabolism, accumulation of large vacuoles (former endosomes) carrying non-digested cargo. Some giant channels allow for the leakage of lysosomal proteases into the cytoplasm. Leaked proteases either digest intracellular proteins (necrosis) or activate apoptosis-related cytoplasmic proteases, which initiate “programmed” cell death. In healthy individuals, the probability of such leakage is extremely low, so induced neuronal death is not reaching critical level, which is needed for the development of significant neuronal loss as is observed in patients with Alzheimer's disease.

FIG. 10. Part of amino acid sequence of beta-amyloid including channel-forming fragment Aβ₂₅₋₃₅ and non-channel-forming fragment Aβ₂₂₋₃₅.

Channel-forming fragment has mostly non-polar amino acids and only one charged amino acid lysin (K), so the overall charge of the peptide is positive. In contrast, longer peptide Aβ₂₂₋₃₅ has two additional charged amino acids—both negatively charged. All genetic mutations which affect this region and are known cause familial types of Alzheimer's disease include the removal of at least of one negative charge. Removal of one negatively charged amino acid increases the probability of the formation of fragments which carry prerequisites of channel-formation: made mostly of non-polar amino acids, able to form beta-sheet structure, and carrying one positively charged amino acid, promoting the interaction with negatively charged membranes.

FIG. 11. Endogenous proteases easily produce channel-forming peptides in mutation-carrying patients.

C-terminal part of full-length peptide Aβ₁₋₄₂ is shown. The peptide is synthesized by digestion of Amyloid Precursor Protein (APP) by β- and γ-secretase. However, there is an alternative processing pathway which includes α-secretase.
A. The sites where APP is cut by α- and γ-secretases are shown. In case of Uppsala deletion, six amino acids appear to be absent (positions 19-24 in the Aβ₁₋₄₂ peptide, or 690-695 in the APP). The location of the deletion is shown.
B. The fragment which is formed after complete digestion by α- and γ-secretases is compared with channel-forming fragment Aβ₂₂₋₃₅. Both are made mostly of non-polar amino acids, able to form beta-sheet structure, and carry one positively charged amino acid, promoting the interaction with negatively charged membranes.

FIG. 12. Classification of Alzheimer's disease based on biochemical process which leads to the beta-amyloid-induced activation of proteolytic damage of neurons and the development and the progression of Alzheimer's disease.

As shown at the FIG. 9, terminal cellular phenomenon defining Alzheimer's disease is neuronal death through apoptosis or necrosis. Four steps leading to the appearance of active proteases in the cytoplasm are the basis for this classification.

Alzheimer's disease Type I. Increased uptake of Aβ due to the intense cellular uptake is the first option which is the etiology of. Due to dramatically increased availability of beta-amyloid to degradation enzymes, increased production of channel-forming fragments leads to the faster neuronal death. The comparison of distributions of major biomarkers data in patients with cognitive impairments (including early- and late-onset AD) and subjects with NC demonstrates that typical late-onset AD can be considered type I (see FIG. 3). In contrast, patients with early-onset AD do not have increased uptake, so they can belong to one of next three types within this classification.

Alzheimer's disease Type II. In contrast to AD type I, the uptake is not increased. However, lysosomal proteolytic activity is disbalanced in favor of endoproteases due to activation of rate of production of endoproteolytic products. Such disbalance results in increased production of channel-forming fragments and leads to faster neuronal death. As was discussed at the FIG. 10, familial types of AD which are associated with mutations in the gene coding amyloid precursor protein, have amino acid substitutions in very specific areas of this protein—removing negatively charged amino acids just outside beta-sheet-forming region increase the production of peptides which 1) can form beta-sheet, 2) carries positive charge, 3) contain mostly hydrophobic amino acids (see FIG. 11)—all features required for the formation of amyloid channels. Increased of production of channel-forming fragments is the feature of AD Type II.

Alzheimer's Type DD (degradation deficiency). The concentration of channel-forming fragments depends on the rate of their formation, but also is influenced by the rate of their farther degradation. If the activity of exoproteases, which are responsible for the digestion of short fragments is decreased, the concentration of toxic proteolytic intermediates increases, and so does the rate of neuronal death. Essentially, this type is about disbalance of proteolytic activity in favor of endoproteases, but unlike AD Type II, due to the decrease of exoproteolytic activity. The example of this phenomenon is shown at the FIG. 15—in low concentrations, anti-chymotrypsin mostly inhibits endoproteolytic activity and ameliorates beta-amyloid toxicity. However, in higher concentration, same inhibitor becomes active towards exoproteases, and its protective action against beta-amyloid toxicity disappears. Similarly, unlike amyloid-protecting aprotinin, which inhibits mostly serine proteases (such as trypsin) which exhibit significant endoproteolytic activity (see FIG. 14), leupeptin, which is more active against cysteine proteases (such as cathepsin B) which exhibit mostly exoproteolytic activity, promotes beta-amyloid toxicity (see FIG. 14).

Alzheimer's Type DS (damage sensitivity). It is obvious that channels are formed with some frequency. However, most likely, not every channel formation event results in host cell death. Firstly, formed channel could be too small for leaking proteases. Without leaking digestive enzymes, disfunctional lysosome can affect normal function of the neuron, but does not necessarily kill the host cell. Secondly, damaged lysosome can be repaired or recycled. Thirdly, leaking lysosomal proteases are inhibited by cytoplasmic inhibitors, such as cystatins, and therefore, the cell could be protected from fatal outcome. According to common sense, same number of formed channels can have no tissue-level consequences in subjects with high resistance, while result in noticeable neuronal death in subjects with low resistance to this outcome. While we cannot offer examples of this mechanism, it should be considered as potentially possible.

It is important to acknowledge, that the progression of AD in a specific patient can be the result of a combination of various mechanisms.

FIG. 13. Alzheimer's disease of various etiologies can be treated by one class of agents.

A: The summary of the mechanisms mediating different types of AD. Please, note that insufficiency of exoproteolytic activity will allow more beta-amyloid to be digested into toxic fragments (Type DD connects to two arrows at the graph).

B: All four described types of AD can be prevented or treated by the inhibition of exoproteases responsible for the formation of toxic channel-forming beta-amyloid fragments, because all degradation of endocytosed beta-amyloid results in the formation of non-toxic products without toxic intermediaries. Due to a significant overlap in activity of different enzymes (the redundancy is needed for effective digestion of various nutrients), we expect that it is possible to achieve medical progress without jeopardizing normal catabolism in the brain, as well as at the system level.

FIG. 14. Selective inhibition of protease prevents apoptosis induced by beta-amyloid (adapted from (Frautschy, Horn et al. 1998)).

Rats were chronically infused intracerebroventricularly with beta-amyloid. Animals were treated either with aprotinin (A) or leupeptin (B), inhibitors of proteases. After the sacrifice, the brains were sectioned and stained for TUNEL, the marker of apoptosis. Aprotinin is more specific towards serine proteases, which are known to have significant endo-proteolytic activity, while leupeptine inhibits wider range of proteases, including cathepsins (such as cathepsin B) with characteristic exopeptidase activity.

Leupeptin tends to increase apoptosis by itself, while aprotinin tends to decrease even spontaneously occurring apoptosis. Chronic infusion of Aβ increases the expression of apoptotic marker TUNEL in the brain. Non-selective inhibition of proteolytic activity with simultaneous i.c.v. infusion of leupeptin promoted apoptosis and an accumulation of extracellular and intracellular Aβ immunoreactivity. In contrast, more selective serine protease inhibitor aprotinin prevented Aβ-induced apoptosis and did not exacerbate intracellular accumulation of amyloid. Protective effect of aprotinin in the absence of promotion of amyloid accumulation demonstrates the possibility to provide the protection without promoting accumulation of amyloid deposits (even though we believe that amyloid deposits themselves are harmless, this point of view is not universally shared).

In the manuscript, which contains this data, the effects are considered in the context of how each inhibitor promotes accumulation of amyloid deposits (leupeptin increases deposits, so promotes the toxicity, while aprotinin does not promote depositions, so does not cause toxicity). There was no connection to toxicity mechanisms related to the formation of channel-forming fragments.

FIG. 15. α1-Anti-chymotrypsin (ACT) dose-dependently inhibit cellular toxicity of beta-amyloid (adapted from (Schubert 1997)).

Two cell lines (rat primary cortical cultures and 15 cells derived from pancreatic islets of Langerhans) were exposed to various concentrations of beta-amyloid in the presence of various concentrations of α1-anti-chymotrypsin, natural serine peptidase inhibitor (serpin). Cell survival was measured using MTT assay.

Beta-amyloid induced drop in cell viability, which was prevented by α1-anti-chymotrypsin. In some conditions, the protection could reach 100%. The optimal dose for inhibition of amyloid toxicity was similar in tests with different concentrations of Aβ but depended on the cell strain: in 15 cells it was 10⁻⁷M, in primary cortical cultures—10⁻⁸M. However, further increase of ACT concentration leads to disappearance of protection (as we discuss in this text, it is due to non-specific inhibition of wider range of proteases). In this manuscript, the author directly challenges the question that anti-proteolytic action is important for the protection, but can not find mechanistic interpretation, and therefore, is not able to reach conclusions about pharmaceutical prospects of new class of pharmacological agents discovered by us.

FIG. 16. Death of pancreatic beta-cells in diabetes Type II can be mediated by a mechanism mediating amyloid degradation toxicity.

Diabetes Type II is characterized by progressive death of beta-cells of islets of Langerhans. These cells are best known due their production of insulin—blood sugar controlling hormone. However, same cells also produce amylin or islet amyloid polypeptide (IAPP). Amylin is amyloidogenic protein and is found in pancreatic tissue of patients with diabetes Type II. It progressively replaces beta-cells in the tissues. Importantly, it is known that amylin can form membrane channels, but unlike Alzheimer's disease, this phenomenon did not catch any significant interest as a druggable process, yet.

The similarity between potentially channel-forming fragments of beta-amyloid and amylin is shown as a table. Each amino acid is labeled according to the physical properties and involvement into beta-sheet formation. It is clear that amylin contains amino acid sequence which is ready to form amyloid membrane channel after appropriate digestion.

FIG. 17. α1-Anti-chymotrypsin (ACT) dose-dependently inhibit cellular toxicity of amylin (adapted from (Schubert 1997)).

15 cells derived from pancreatic islets of Langerhans were exposed to amylin in the presence of various concentrations of α1-anti-chymotrypsin, natural serine peptidase inhibitor (serpin). Cell survival was measured using MTT assay.

Amylin induced drop in cell viability, which was prevented by α1-anti-chymotrypsin. Unlike effects against beta-amyloid (see FIG. 15), the protection against amylin was not reaching 100% (based on the amount of presented information in the manuscript, it could be suggested that the author actually placed much less attention to this part of the study). Similar to the case of anti-beta-amyloid protection, ACT demonstrated optimal dose, further increase of ACT concentration significantly decreased the protective effect.

FIG. 18. Amino acid sequence of superoxide dismutase (SOD) which is associated with the development of ALS (amyotrophic lateral sclerosis).

Like Alzheimer's disease and diabetes type II, ALS is characterized by the death of specific cell type. Dying cells in all degenerative diseases exhibit several important similarities such as lysosomal disfunction, increased production of reactive oxygen species linked to improper recycling of mitochondria. One of hypothetical mechanisms involved into the development of ALS includes superoxide dismutase, key enzyme needed for protection against damaging intermediaries formed by active oxygen. SOD is the protein which is considered misfolding (forming aggregates despite being soluble initially).

The schematic of biochemical events described at the FIGS. 8 and 9 (amyloid degradation toxicity) can be traced to the ALS when amino acid sequence of superoxide dismutase, the protein which is believed to be involved into the pathogenesis of ALS, is considered. The molecule of SOD contains 154 amino acids, which form two pairs of beta-sheets—top and bottom beta-sheets. Each beta-sheet is formed by four strands. The amino acid sequence is shown, the sequences involved into each strand of beta-sheet and the charges of amino acids which carry either positive or negative charge.

FIG. 19. Superoxide dismutase (SOD) contains amino acid sequences which are potentially membrane channel-forming.

At least two fragments of amino acid sequence of SOD resemble the structure of membrane channel-forming fragment of beta-amyloid. Both fragments consist mostly by non-polar amino acids, form beta-sheet, and have a single positively charged amino acid in the sequence.

### DETAILED DESCRIPTION OF THE INVENTION

According to our amyloid degradation toxicity theory, the cytotoxic effects of amyloidogenic peptides are mediated, at least in part, by the formation of membrane channels in cellular membranes. Unlike full-length amyloid peptides which are not effective in forming channels, some degradation products of said peptides are. Therefore, we claim that the prevention of proteolytic degradation of said peptides is the method to prevent or slow down the development of diseases caused by said peptides. Said prevention is the therapeutic method, which is clearly distinct from other treatment options known so far, such as the decrease of production of amyloid peptides from pro-peptides, inactivation of full-length peptides by antibodies, or ameliorating consequences of ion transport disturbance.

We claim that the use of chemical entities which selectively inhibit proteases digesting amyloid peptides into fragments which form membrane channels can be achieved without significant effect on overall metabolism of amyloid peptide and without excessive production of extracellular deposits of said peptides which can be histochemically stained as amyloid in postmortem specimens in patients. Main embodiment of this invention is a method to treat neurodegenerative disease, such as Alzheimer's disease using inhibitors of proteases.

Among embodiments of this invention is the method to select chemical entities, which are effective in the treatment of said diseases using the method invented by us previously ((Zaretsky and Zaretskaia 2020), patent pending). The etiology of neurodegenerative diseases such as Alzheimer's disease is in the formation of peptide fragments which are able to form channels. The method considers mixing biological samples (such as purified enzymes or homogenates of tissues) with protein of interest (such as beta-amyloid peptide in studies of Alzheimer's disease or superoxide dismutase in studies of amyotrophic lateral sclerosis) and collecting samples from the mixture after desired times. Degradation of amyloid protein by proteases in the sample results in the production of fragments. If fragments are able to form membrane channels, the presence of channel-forming units is tested using test liposomes and flowmetric technique to measure permeabilization of membranes (Zaretsky and Zaretskaia 2020).

We expect that high-throughput testing will reveal multiple chemical entities which can be used to treat amyloidogenic diseases using invented method, with special interest that some of these effective chemical entities may be already approved by FDA or other regulatory agency as the drugs with other indications (such as aprotinin). Availability of medicines, which are effective with off-label use, may be a fast pathway to deliver life-saving treatments to patients.

Various ways to suppress the enzymatic proteolytic activity can be suggested, such as the use of neutralizing anti-enzymatic antibodies, genetic modification of synthesis of appropriate enzymes, the modification of synthesis of endogenous inhibitors or the delivery of exogenous synthetic mechanisms to produce appropriate inhibitors in situ.

### EXAMPLES OF HOW THE INVENTION WILL BE USED

### Example 1

New pharmacologic class of drugs to treat neurodegenerative diseases is established. The criterion to belong to this new class is the drug's ability to prevent the degradation of beta-amyloid into fragments which are able to form membrane ion channels.

### Example 2

The method for studying degradation of proteins into fragments which are able to form membrane channels will be used to study molecular mechanisms involved in the progression of neurodegenerative diseases, such as Alzheimer's disease. One of applications is to screen enzymes responsible for said degradation.

Liposomes with embedded ion-sensitive probe are used as a test system. Liposomes are extruded from phosphatidylserine containing membrane probe (i.e. DiD) in a calcium-free buffer containing ion-sensitive probe (i.e. Fluo-4) and volume probe (i.e. dextran-tetramethylrhodamin) or membrane probe (DiD). Extravesicular probes are cleared using either centrifugation, or dialysis. After the addition of calcium, the intravesicular calcium-sensing probe remain non-fluorescent because membranes are not permeable to calcium. If membrane channels are formed, calcium enters permeable liposomes, saturates the calcium-sensing dye, so the liposome becomes fluorescent. The number of fluorescent liposomes is an estimate of the number of channel-forming units in the solution.

To assay channel-forming unit formation, full-length beta-amyloid is mixed with sample that possesses protein-degrading activity. After incubation in desired conditions, the mixture is added to the suspension of liposomes, and analyzed on the flow cytometer. The number of formed channels is estimated from the number of permeabilized liposomes.

The sample with protein-degrading activity could be a solution of protease (recombinant or purified from a natural source), homogenate of a tissue, lysate of organelle preparation (for example, isolated lysosomes or mitochondria).

### Example 3

The method for high throughput testing of chemical entities for an ability to inhibit enzymes degrading proteins into fragments which are able to form membrane channels will be used to find drug candidates to treat neurodegenerative diseases, such as Alzheimer's disease.

This is an extension of the technique described in the Example 2 using a particular enzyme that was validated as a key player involved in the peptide channel-mediated cellular toxicity.

Chemical entity that significantly decreases the formation of membrane channels is considered effective against channel-mediated permeabilization of membranes.

### REFERENCES CITED

Abramov, A. Y., L. Canevari and M. R. Duchen (2004). “Calcium signals induced by amyloid β peptide and their consequences in neurons and astrocytes in culture.” Biochimica et Biophysica Acta (BBA)—Molecular Cell Research 1742(1): 81-87.
Aksenov, M. Y., M. V. Aksenova, J. M. Carney and D. A. Butterfield (1996). “Alpha 1-antichymotrypsin interaction with A beta (1-42) does not inhibit fibril formation but attenuates the peptide toxicity.” Neurosci Lett 217(2-3): 117-120.
Alarcon, J. M., J. A. Brito, T. Hermosilla, I. Atwater, D. Mears and E. Rojas (2006). “Ion channel formation by Alzheimer's disease amyloid beta-peptide (Abeta40) in unilamellar liposomes is determined by anionic phospholipids.” Peptides 27(1): 95-104.
Ariosa, A. R. and D. J. Klionsky (2016). “Autophagy core machinery: overcoming spatial barriers in neurons.” Journal of molecular medicine (Berlin, Germany) 94(11): 1217-1227.
Arispe, N., J. C. Diaz and O. Simakova (2007). “Aβ ion channels. Prospects for treating Alzheimer's disease with Aβ channel blockers.” Biochimica et Biophysica Acta (BBA)—Biomembranes 1768(8): 1952-1965.
Arispe, N., H. B. Pollard and E. Rojas (1993). “Giant multilevel cation channels formed by Alzheimer disease amyloid beta-protein [A beta P-(1-40)] in bilayer membranes.” Proceedings of the National Academy of Sciences of the United States of America 90(22): 10573-10577.
Arispe, N., H. B. Pollard and E. Rojas (1994). “The ability of amyloid beta-protein [A beta P (1-40)] to form Ca2+ channels provides a mechanism for neuronal death in Alzheimer's disease.” Annals of the New York Academy of Sciences 747: 256-266.
Arispe, N., H. B. Pollard and E. Rojas (1994). “beta-Amyloid Ca(2+)-channel hypothesis for neuronal death in Alzheimer disease.” Molecular and cellular biochemistry 140(2): 119-125.
Arispe, N., H. B. Pollard and E. Rojas (1996). “Zn2+ interaction with Alzheimer amyloid beta protein calcium channels.” Proceedings of the National Academy of Sciences 93(4): 1710-1715.
Arispe, N., E. Rojas and H. B. Pollard (1993). “Alzheimer disease amyloid beta protein forms calcium channels in bilayer membranes: blockade by tromethamine and aluminum.” Proceedings of the National Academy of Sciences of the United States of America 90(2): 567-571.
Benz, R. (1985). “Porin from bacterial and mitochondrial outer membranes.” CRC Crit Rev Biochem 19(2): 145-190.
Blass, J. P. (2000). “The mitochondrial spiral. An adequate cause of dementia in the Alzheimer's syndrome.” Ann NY Acad Sci 924: 170-183.
Bode, D. C., M. D. Baker and J. H. Viles (2017). “Ion Channel Formation by Amyloid-β42 Oligomers but Not Amyloid-β40 in Cellular Membranes.” 292(4): 1404-1413.
Boland, B., A. Kumar, S. Lee, F. M. Platt, J. Wegiel, W. H. Yu and R. A. Nixon (2008). “Autophagy induction and autophagosome clearance in neurons: relationship to autophagic pathology in Alzheimer's disease.” J Neurosci 28(27): 6926-6937.
Busche, M. A., X. Chen, H. A. Henning, J. Reichwald, M. Staufenbiel, B. Sakmann and A. Konnerth (2012). “Critical role of soluble amyloid-beta for early hippocampal hyperactivity in a mouse model of Alzheimer's disease.” Proc Natl Acad Sci USA 109(22): 8740-8745.
Busche, M. A. and A. Konnerth (2016). “Impairments of neural circuit function in Alzheimer's disease.” Philosophical transactions of the Royal Society of London. Series B, Biological sciences 371(1700): 20150429.
Chafekar, S. M., F. Baas and W. Scheper (2008). “Oligomer-specific Aβ toxicity in cell models is mediated by selective uptake.” Biochimica et Biophysica Acta (BBA)—Molecular Basis of Disease 1782(9): 523-531.
Chen, J. X. and S. D. Yan (2007). “Amyloid-beta-induced mitochondrial dysfunction.” Journal of Alzheimer's disease: JAD 12(2): 177-184.
Chen, J. X. and S. S. Yan (2010). “Role of mitochondrial amyloid-beta in Alzheimer's disease.” J Alzheimers Dis 20 Suppl 2: S569-578.
Cherra, S. J., 3rd and C. T. Chu (2008). “Autophagy in neuroprotection and neurodegeneration: A question of balance.” Future Neurol 3(3): 309-323.
Cline, E. N., M. A. Bicca, K. L. Viola and W. L. Klein (2018). “The Amyloid-β Oligomer Hypothesis: Beginning of the Third Decade.” Journal of Alzheimer's disease: JAD 64(s1): S567-S610.
Colombini, M. (2012). “VDAC structure, selectivity, and dynamics.” Biochimica et biophysica acta 1818(6): 1457-1465.
Demetrius, L. A., P. J. Magistretti and L. Pellerin (2015). “Alzheimer's disease: the amyloid hypothesis and the Inverse Warburg effect.” Frontiers in physiology 5: 522-522.
Diaz, J. C., O. Simakova, K. A. Jacobson, N. Arispe and H. B. Pollard (2009). “Small molecule blockers of the Alzheimer Abeta calcium channel potently protect neurons from Abeta cytotoxicity.” Proc Natl Acad Sci USA 106(9): 3348-3353.
Dominguez-Prieto, M., A. Velasco, A. Tabernero and J. M. Medina (2018). “Endocytosis and Transcytosis of Amyloid-β Peptides by Astrocytes: A Possible Mechanism for Amyloid-β Clearance in Alzheimer's Disease.” Journal of Alzheimer's Disease 65: 1109-1124.
Drews, A., J. Flint, N. Shivji, P. Jonsson, D. Wirthensohn, E. De Genst, C. Vincke, S. Muyldermans, C. Dobson and D. Klenerman (2016). “Individual aggregates of amyloid beta induce temporary calcium influx through the cell membrane of neuronal cells.” Scientific Reports 6: 31910.
Durell, S. R., H. R. Guy, N. Arispe, E. Rojas and H. B. Pollard (1994). “Theoretical models of the ion channel structure of amyloid beta-protein.” Biophys J 67(6): 2137-2145.
Eckert, A., K. Schmitt and J. Gotz (2011). “Mitochondrial dysfunction—the beginning of the end in Alzheimer's disease? Separate and synergistic modes of tau and amyloid-β toxicity.” Alzheimers Res Ther 3(2): 15.
Erickson, H. P. (2009). “Size and shape of protein molecules at the nanometer level determined by sedimentation, gel filtration, and electron microscopy.” Biological procedures online 11: 32-51.
Frautschy, S. A., D. L. Horn, J. J. Sigel, M. E. Harris-White, J. J. Mendoza, F. Yang, T. C. Saido and G. M. Cole (1998). “Protease inhibitor coinfusion with amyloid beta-protein results in enhanced deposition and toxicity in rat brain.” The Journal of neuroscience: the official journal of the Society for Neuroscience 18(20): 8311-8321.
Glabe, C. G. (2006). “Common mechanisms of amyloid oligomer pathogenesis in degenerative disease.” Neurobiol Aging 27(4): 570-575.
He, C. and D. J. Klionsky (2009). “Regulation mechanisms and signaling pathways of autophagy.” Annu Rev Genet 43: 67-93.
Heckmann, B. L., B. J. W. Teubner, B. Tummers, E. Boada-Romero, L. Harris, M. Yang, C. S. Guy, S. S. Zakharenko and D. R. Green (2019). “LC3-Associated Endocytosis Facilitates β-Amyloid Clearance and Mitigates Neurodegeneration in Murine Alzheimer's Disease.” Cell 178(3): 536-551.e514.
Hirakura, Y., M. C. Lin and B. L. Kagan (1999). “Alzheimer amyloid abeta1-42 channels: effects of solvent, pH, and Congo Red.” J Neurosci Res 57(4): 458-466.
Jang, H., F. T. Arce, S. Ramachandran, R. Capone, R. Azimova, B. L. Kagan, R. Nussinov and R. Lal (2010). “Truncated beta-amyloid peptide channels provide an alternative mechanism for Alzheimer's Disease and Down syndrome.” Proceedings of the National Academy of Sciences of the United States of America 107(14): 6538-6543.
Jang, H., L. Connelly, F. T. Arce, S. Ramachandran, R. Lal, B. L. Kagan and R. Nussinov (2013). “Alzheimer's disease: which type of amyloid-preventing drug agents to employ?” Phys Chem Chem Phys 15(23): 8868-8877.
Ji, Z. S., R. D. Miranda, Y. M. Newhouse, K. H. Weisgraber, Y. Huang and R. W. Mahley (2002). “Apolipoprotein E4 potentiates amyloid beta peptide-induced lysosomal leakage and apoptosis in neuronal cells.” J Biol Chem 277(24): 21821-21828.
Jin, S., N. Kedia, E. Illes-Toth, I. Haralampiev, S. Prisner, A. Herrmann, E. E. Wanker and J. Bieschke (2016). “Amyloid-β(1-42) Aggregation Initiates Its Cellular Uptake and Cytotoxicity.” J Biol Chem 291(37): 19590-19606.
Knauer, M. F., B. Soreghan, D. Burdick, J. Kosmoski and C. G. Glabe (1992). “Intracellular accumulation and resistance to degradation of the Alzheimer amyloid A4/beta protein.” Proc Natl Acad Sci USA 89(16): 7437-7441.
Lee, S., Y. Sato and R. A. Nixon (2011). “Lysosomal proteolysis inhibition selectively disrupts axonal transport of degradative organelles and causes an Alzheimer's-like axonal dystrophy.” J Neurosci 31(21): 7817-7830.
Lin, H. and N. J. Arispe (2015). “Single-cell screening of cytosolic [Ca(2+)] reveals cell-selective action by the Alzheimer's Aβ peptide ion channel.” Cell Stress Chaperones 20(2): 333-342.
Lin, H., R. Bhatia and R. Lal (2001). “Amyloid beta protein forms ion channels: implications for Alzheimer's disease pathophysiology.” Faseb j 15(13): 2433-2444.
Lin, H., Y. J. Zhu and R. Lal (1999). “Amyloid beta protein (1-40) forms calcium-permeable, Zn2+-sensitive channel in reconstituted lipid vesicles.” Biochemistry 38(34): 11189-11196.
Lin, M.-c. A. and B. L. Kagan (2002). “Electrophysiologic properties of channels induced by Abeta25-35 in planar lipid bilayers.” Peptides 23(7): 1215-1228.
Ma, J., H. B. Brewer, Jr. and H. Potter (1996). “Alzheimer A beta neurotoxicity: promotion by antichymotrypsin, ApoE4; inhibition by A beta-related peptides.” Neurobiol Aging 17(5): 773-780.
Marshall, K. E., D. M. Vadukul, K. Staras and L. C. Serpell (2020). “Misfolded amyloid-β-42 impairs the endosomal-lysosomal pathway.” Cell Mol Life Sci 77(23): 5031-5043.
Micelli, S., D. Meleleo, V. Picciarelli and E. Gallucci (2004). “Effect of sterols on beta-amyloid peptide (AbetaP 1-40) channel formation and their properties in planar lipid membranes.” Biophysical journal 86(4): 2231-2237.
Mindell, J. A. (2012). “Lysosomal acidification mechanisms.” Annu Rev Physiol 74(1): 69-86.
Mirzabekov, T., M. C. Lin, W. L. Yuan, P. J. Marshall, M. Carman, K. Tomaselli, I. Lieberburg and B. L. Kagan (1994). “Channel formation in planar lipid bilayers by a neurotoxic fragment of the beta-amyloid peptide.” Biochemical and biophysical research communications 202(2): 1142-1148.
Mroczko, B., M. Groblewska, A. Litman-Zawadzka, J. Kornhuber and P. Lewczuk (2018). “Cellular Receptors of Amyloid β Oligomers (APOs) in Alzheimer's Disease.” International journal of molecular sciences 19(7): 1884.
Naldi, M., J. Fiori, M. Pistolozzi, A. F. Drake, C. Bertucci, R. Wu, K. Mlynarczyk, S. Filipek, A. De Simone and V. Andrisano (2012). “Amyloid β-peptide 25-35 self-assembly and its inhibition: a model undecapeptide system to gain atomistic and secondary structure details of the Alzheimer's disease process and treatment.” ACS chemical neuroscience 3(11): 952-962.
Nelson, B. D. and F. Kabir (1986). “The role of the mitochondrial outer membrane in energy metabolism of tumor cells.” Biochimie 68(3): 407-415.
Nixon, R. A., J. Wegiel, A. Kumar, W. H. Yu, C. Peterhoff, A. Cataldo and A. M. Cuervo (2005). “Extensive involvement of autophagy in Alzheimer disease: an immuno-electron microscopy study.” J Neuropathol Exp Neurol 64(2): 113-122.
Parodi, J., F. J. Sepulveda, J. Roa, C. Opazo, N. C. Inestrosa and L. G. Aguayo (2010). “Beta-amyloid causes depletion of synaptic vesicles leading to neurotransmission failure.” J Biol Chem 285(4): 2506-2514.
Pollard, H. B., N. Arispe and E. Rojas (1995). “Ion channel hypothesis for Alzheimer amyloid peptide neurotoxicity.” Cellular and molecular neurobiology 15(5): 513-526.
Pollard, H. B., E. Rojas and N. Arispe (1993). “A new hypothesis for the mechanism of amyloid toxicity, based on the calcium channel activity of amyloid beta protein (A beta P) in phospholipid bilayer membranes.” Annals of the New York Academy of Sciences 695: 165-168.
Rhee, S. K., A. P. Quist and R. Lal (1998). “Amyloid beta protein-(1-42) forms calcium-permeable, Zn2+-sensitive channel.” The Journal of biological chemistry 273(22): 13379-13382.
Rizzuto, R., D. De Stefani, A. Raffaello and C. Mammucari (2012). “Mitochondria as sensors and regulators of calcium signalling.” Nature Reviews Molecular Cell Biology 13(9): 566-578.
Schubert, D. (1997). “Serpins inhibit the toxicity of amyloid peptides.” Eur J Neurosci 9(4): 770-777.
Sharoar, M. G., X. Hu, X.-M. Ma, X. Zhu and R. Yan (2019). “Sequential formation of different layers of dystrophic neurites in Alzheimer's brains.” Molecular Psychiatry 24(9): 1369-1382.
Shirwany, N. A., D. Payette, J. Xie and Q. Guo (2007). “The amyloid beta ion channel hypothesis of Alzheimer's disease.” Neuropsychiatr Dis Treat 3(5): 597-612.
Simmons, M. A. and C. R. Schneider (1993). “Amyloid beta peptides act directly on single neurons.” Neurosci Lett 150(2): 133-136.
Smith, L. M. and S. M. Strittmatter (2017). “Binding Sites for Amyloid-β Oligomers and Synaptic Toxicity.” Cold Spring Harbor perspectives in medicine 7(5): a024075.
Teplow, D. B. (2013). “On the subject of rigor in the study of amyloid β-protein assembly.” Alzheimers Res Ther 5(4): 39.
Tizon, B., E. M. Ribe, W. Mi, C. M. Troy and E. Levy (2010). “Cystatin C protects neuronal cells from amyloid-beta-induced toxicity.” J Alzheimers Dis 19(3): 885-894.
Wesen, E., G. D. M. Jeffries, M. Matson Dzebo and E. K. Esbjorner (2017). “Endocytic uptake of monomeric amyloid-β peptides is clathrin- and dynamin-independent and results in selective accumulation of Aβ(1-42) compared to Aβ(1-40).” Scientific reports 7(1): 2021-2021.
Yang, A. J., D. Chandswangbhuvana, L. Margol and C. G. Glabe (1998). “Loss of endosomal/lysosomal membrane impermeability is an early event in amyloid Abeta1-42 pathogenesis.” J Neurosci Res 52(6): 691-698.
Yin, Z., C. Pascual and D. J. Klionsky (2016). “Autophagy: machinery and regulation.” Microbial cell (Graz, Austria) 3(12): 588-596.
Zaretsky, D. and M. Zaretskaia (2020). “Multiscale Analysis of the Amyloid Degradation Toxicity Hypothesis of Alzheimer's Disease.” Preprints.org: 2020120813; doi: 2020120810.2020120944/preprints2020202012.2020120813.v2020120811.
Zaretsky, D. V. and M. Zaretskaia (2020). “Degradation Products of Amyloid Protein: Are They The Culprits?” Curr Alzheimer Res 17(10): 869-880.
Zaretsky, D. V. and M. V. Zaretskaia (2020). “Flow cytometry method to quantify the formation of beta-amyloid membrane ion channels.” Biochim Biophys Acta Biomembr: 183506.
Zaretsky, D. V. and M. V. Zaretskaia (2021). “Mini-review: Amyloid degradation toxicity hypothesis of Alzheimer's disease.” Neurosci Lett 756: 135959.
Zaretsky, D. V., M. V. Zaretskaia and Y. I. Molkov (2021). “Membrane channel hypothesis of lysosomal permeabilization by beta-amyloid.” Neurosci Lett: 136338.

1. The method of treatment of degenerative diseases using an inhibition of enzymes degrading peptides or proteins into fragments which are able to form membrane channels in cellular membranes.

2. The method of claim 1, wherein said inhibition is achieved by an introduction of a molecule with inhibitory activity (such as giving the patient a medicine).

3. The method of claim 1, wherein said inhibition is achieved by a modification of synthesis of such enzymes.

4. The method of claim 1, wherein said inhibition is made by a macromolecule or a mix of macromolecules which can bind such enzymes (such as antibodies or affibodies).

5. The method of claim 1, wherein said inhibition of said enzymes is achieved by increased synthesis of endogenous inhibitor or inhibitors.

6. The method of claim 1, wherein the inhibition of such enzymes is achieved by the introduction of a synthesis of inhibitor or inhibitors in the organism (such as viral delivery of nucleic acid sequence for the peptide inhibitor).

7. The method of claim 1, wherein the disease is Alzheimer disease or other degenerative disease caused by peptides from the family of beta-amyloid (full-length or its fragments, native or including mutations, occurring naturally, or induced artificially).

8. The method of claim 1, wherein the disease is Parkinson's disease or other degenerative disease, caused by peptides from the family of alpha-synuclein (full-length or its fragments, native or including mutations, occurring naturally, or induced artificially).

9. The method of claim 1, wherein the disease is ALS (amyotrophic lateral sclerosis) or other degenerative disease, caused by peptides from the family of superoxide dismutase (full-length or its fragments, native or including mutations, occurring naturally, or induced artificially).

10. The method of claim 1, wherein the disease is ALS (amyotrophic lateral sclerosis) or other degenerative disease, caused by peptides from the family of TDP-43 (full-length or its fragments, native or including mutations, occurring naturally, or induced artificially).

11. The method of claim 1, wherein the disease is Alzheimer's disease or other degenerative disease, caused by peptides from the family of tau protein (full-length or its fragments, native or including mutations, occurring naturally or induced artificially, phosphorylated or not).

12. The method of claim 1, wherein the disease is diabetes mellitus type II or other degenerative disease, caused by peptides from the family of amylin (full-length or its fragments, native or including mutations, occurring naturally, or induced artificially).

13. The method of claim 1, wherein the disease is Huntington's disease or other degenerative disease, caused by peptides from the family of Huntingtin's protein (full-length or its fragments, native or including mutations, occurring naturally, or induced artificially).

14. The method of claim 1, wherein the disease is prion disease or other disease, caused by peptides from the family of prions (full-length or its fragments, native or including mutations, occurring naturally or induced artificially).
